(19) United States
(12) Patent Application Publication
(10) Pub. No.: US 2009/0247472 A1
(43) Pub. Date: Oct. 1, 2009

(54) TYPE 1, 4-NAPHTOQUINONE COMPOUNDS, COMPOSITIONS COMPRISING THEM AND USE OF THESE COMPOUNDS AS ANTI-CANCER AGENTS

(75) Inventors: Gaelle Depierre, Saint Seurin De Bourg (FR); Jean Dessolin, Merignac (FR); Michel Laguerre, Leognan (FR); Claude Commandeur, Talence (FR)

Correspondence Address: BACHMAN & LAPOINTE, P.C., 900 CHAPEL STREET, SUITE 1201, NEW HAVEN, CT, 06510, US

(73) Assignees: FLUOFARMA, Pessac (FR); UNIVERSITE BORDEAUX 1, Talence Cedex (FR); CENTRE NATIONAL DE LA RECHERCHE SCIENTIFIQUE, Paris Cedex (FR)

(21) Appl. No.: 12/298,307

(22) PCT Filed: Apr. 25, 2007
(86) PCT No.: PCT/FR07/00703

§ 371 Date: May 20, 2009

(30) Foreign Application Priority Data
Apr. 25, 2006 (FR) 0651461

(51) Int. Cl. A61K 38/05; C07C 237/52; A61K 31/137; C07C 211/30; C07C 215/54; A61P 35/00

(52) U.S. Cl. 514/19; 564/155; 514/654; 564/378; 564/360; 514/653

(57) ABSTRACT

This invention relates to compounds with the formula (I) given below or one of their pharmaceutically acceptable salts, as a medicine; Formula (I) of pharmaceutical compositions comprising one or more compounds with Formula (I) as active constituent, use of compounds with Formula (I) for the preparation of compositions designed to prevent or treat at least one illness involving an abnormal cellular proliferation, pro-apoptotic compositions and/or anti-proliferative compositions comprising at least one compound with Formula (I)/and the use of compounds with formula (I) as pro-apoptotic and/or anti-proliferative agents.

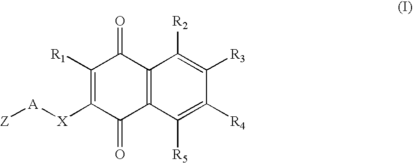

### PRIORITY

The present invention claims priority right over French Patent Application No 06/51461 filed on 25 Apr. 2006. This application is hereby incorporated, by reference, in its entirety.

### DESCRIPTION

1. Technical Field

The present invention concerns the field of the prevention and treatment of diseases involving abnormal cell proliferation.

It concerns more precisely compounds of the 1,4-naphthoquinone type, in particular by way of medication, and the use of such compounds in the preparation of pharmaceutical compositions. These pharmaceutical compositions can in particular be intended to prevent or treat disorders involving abnormal cell proliferation, in particular cancer. The invention also concerns pro-apoptotic and/or anti-proliferative compositions comprising compounds of this type, or the use of this type of compound as a pro-apoptotic and/or anti-proliferative agent.

2. Prior Art

Cancer is one of the greatest causes of mortality and consequently one of the most serious public health problems in the world today. Numerous medications have been and are being developed. However, these medications do not make it possible to treat all cases with success. Moreover, the drugs used in the context of chemotherapies may have undesirable secondary effects, and insufficient efficacy and/or specificity of action vis-à-vis cancerous cells.

Among the agents used in anti-cancer therapies, a certain number of agents that induce apoptosis can be cited. This is because cancerous cells are frequently resistant to apoptosis, this programmed cell death phenomenon being inhibited.

Apoptosis, or programmed cell death, is a physiological process essential to the maintenance of tissue homeostasis, and is the mechanism by means of which the organism regulates the quantity of cells necessary for its wellbeing and development.

This process is particularly interesting since, unlike necrosis, it does not exhibit any release of inflammation mediators in the extracellular environment. Thus, in the context of an application in anti-cancer therapy, it may make it possible to cause a “clean death”, by comparison with necrosis, at the tumorous cells.

Among the pro-apoptotic compounds, the peptides of SMAC (second mitochondria activator of caspases) can be cited. However, the peptide fragments of SMAC may cause many problems during use in vivo, for example low bioavailability, excessively rapid degradation and/or excessively high immunogenicity.

There remains a need for novel compounds having anti-cancerous activity, in particular having very good specificity of action towards malignant cells, improved efficacy, at least in certain types of cancers, and/or the secondary effects of which are reduced.

### DESCRIPTION OF THE INVENTION

The inventors have now discovered that the compounds as defined below have anti-cancerous activity, in particular relating to a pro-apoptotic and/or anti-proliferative activity, while at least partly resolving the problems mentioned above.

Without wishing to be bound by any hypothesis, it is possible that the mode of action of the molecules according to the invention is related to the fact that these molecules mimic at least an essential part of SMAC.

Thus, according to a first aspect, an object of the invention is the isolated compounds complying with the following formula (I) or one of its pharmaceutically acceptable salts, by way of medication:

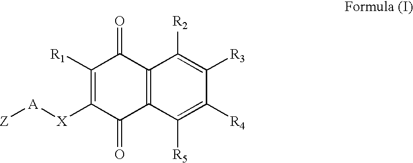

in which:
R₁ represents a hydrogen atom; a halogen atom; a hydroxyl function, possibly substituted, an alkyl, alkene, or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; a nitro function; or an —X′-A′-Z′ function in which;
   (i) X′ represents a divalent radical, in particular chosen from alkyls, alkenes or alkynes, linear, branched or cyclic, substituted or not, chiral or non-chiral, possibly interrupted by a heteroatom,
   (ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, in particular chosen from those described in the work “Protective Groups in Organic Synthesis” by T W Greene, P G M Wuts, Wiley-Interscience, New York, 4^{th} edition, 2007, and
   (iii) Z′ represents an amino acid residue, in particular D or L, natural or synthetic, in particular an α, β or γ amino acid residue, the terminal amine or carboxyl function (ie not linked to X′) and any lateral chemical functions of Z′ being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R2, R3, R4 and R5 represent independently of one another a hydrogen atom; a halogen atom; a hydroxyl function, possibly substituted, an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; or a nitro function;
   or R2 and R3, R3 and R4 and/or R4 and R5 form together a ring or a heterocyclic compound, possibly substituted,
X represents a divalent radical, in particular chosen from alkyls, alkenes or alkynes, linear, branched or cyclic, substituted or not, chiral or non-chiral, possibly interrupted by a heteroatom,
A is —O—, —S—, —NY′—, —SO₂—, —C(═S)—, —CO— or a chemical function such that Z′ and X′ are linked by a bioisosteric bond of the amine function, where Y represents a hydrogen atom or a protective group, in particular chosen from those described in the work “Protective Groups in Organic Synthesis” by T W Greene, P G M Wuts, Wiley-Interscience, New York, 4^{th} edition, 2007, and
Z represents an amino acid residue, in particular D or L, natural or synthetic, in particular an α, β or γ amino acid residue, the terminal amine or carboxyl function (ie not linked to X) and any lateral chemical functions of Z being protected or not, where Z is linked to the X radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z resulting from the reduction of the terminal carboxyl function of the Z amino acid residue;
   in particular X and X′ are, independently of each other, possibly substituted by one or more chemical functions such as a lateral chain of a natural amino acid; C₁₋₆alkyl; C₂₋₆alkene; C₂₋₆alkyne; C₃₋₈cycloalkyl; C₁₋₆heteroalkyl; C₁₋₆haloalkyl; C₆₋₁₀aryl; C₃₋₈heteroaryl; C₅₋₂₀heterocyclic; C₁₋₆alkylC₆₋₁₀aryl; C₁₋₆alkylC₃₋₈heteroaryl; C₁₋₆alkoxy; C₆₋₁₀aryloxy; C₃₋₈heteroalkoxy; C₃₋₈heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2} SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, heterocyclic compound, alkylaryl or alkylheteroaryl group in which the aryl, heteroaryl or heterocyclic radical is possibly substituted; or, when G represents —NR^{G2}—, R^{G1} and R^{G2} conjointly with the nitrogen atom to which they are bonded form a heterocyclic compound or a heteroaryl, possibly substituted,
   in particular the said bioisosteric bond of the amide bond is in accordance with the examples cited in the following articles:
(1) Giannis, A; Kolter, T “Peptidomimetics for Receptor Ligands Discovery Development and Medicinal Perspectives,” Angew Chem, Int Ed Eng, 1993, 32, 1244-1267;
(2) Roark, W; Roth B; Holmes, A; Trivedi, B; Kieft, K; Essenburg; Krausse, B; Stanfield, R “Inhibitors of Acyl-CoA:Cholesterol Acyltransferase (ACAT)-2. Modification of Fatty Acid Anilide ACAT Inhibitors: Bioisosteric Replacement of the Amide Bond,” J Med Chem, 1993, 36, 1662-1668.

These definitions are not limitative and, for example, a substituted amine (secondary, tertiary) can be considered as an isostere of the amide bond for example.

In a particular embodiment, for example, Z is bonded to the X radical via its terminal carboxyl function, and A is —O—, —S— or —NY— where the Y group represents a hydrogen atom or a protective group, in particular chosen from those described in the work “Protective Groups in Organic Synthesis” by T W Greene, P G M Wuts, Wiley-Interscience, New York, 4^{th} edition, 2007. The terminal amine function of Z, and/or any lateral chemical functions of Z, are free or possibly protected.

In a particular embodiment, Z is bonded to the X radical via its terminal amine function, and A is —SO₂—, —C(═S)— or —CO—. The terminal carboxyl function of Z, and/or any lateral chemical functions of Z, are free and possibly protected.

When Z is bonded to the X radical via its terminal carboxyl function, the terminal amine function f Z may be in the form of a quaternary amine salt such as a chlorhydrate, a bromhydrate, a trifluoroacetate (etc). When Z is bonded to the X radical via its terminal carboxyl function, the terminal amine function of Z, as well as any functions carried by the lateral chain of the amino acid residue Z (hydroxyl, amine, guanidine, etc), may be protected by a protective group of the said function such as those described in the work “Protective Groups in Organic Synthesis” by T W Greene, P G M Wuts, Wiley-Interscience, New York, 4^{th} edition, 2007. The terminal amine function of Z, and/or any lateral chemical functions of Z, are free or possibly protected.

When Z is bonded to the X radical via its terminal amine function, the terminal carboxyl function of Z may be in the form of a salt such as a sodium or potassium salt. When Z is bonded to the X radical via its terminal amine function, the terminal carboxyl function of Z, as well as any functions carried by the lateral chain of the amino acid residue Z (hydroxyl, amine, guanidine, etc), may be protected by a protective group of the said function such as those described in the work “Protective Groups in Organic Synthesis” by T W Greene, P G M Wuts, Wiley-Interscience, New York, 4^{th} edition, 2007. The terminal carboxyl function of Z, and/or any lateral chemical functions of Z, are free or possibly protected.

More particularly, the X group represents a divalent radical of the type —(CH₂)ₙ—, in which n is an integer number ranging from 1 to 10 in particular from 2 to 8.

More particularly, the X group represents a branched divalent radical complying with the formula —(CH₂)ₚ—CHRₓ—(CH₂)_{q}— in which p and q are independently of each other an integer number ranging from 0 to 12, and the sum p+q is an integer number ranging from 1 to 12, in particular from 1 to 7, and R_{X} is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₈heteroaryl; C₁₋₆alkoxy; C₆₋₁₀aryloxy; C₃₋₈heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}, —C(═O)—, —S(═O)—, —SO₂—, —C(═O)O—, —C(═O)NR^{G2}—, —OC(═O)—, —NR^{G2}C(—C(═O)0-, —OC(═O)NR^{G2}, —NR^{G2}C(═O)O—, —NR^{G2}C(═O)NR^{G2}, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, heterocyclic compound, alkylaryl or alkylheteroaryl group in which the aryl, heteroaryl or heterocyclic radical is possibly substituted; or, when G represents —NR^{G2}—, R^{G1} and R^{G2} conjointly with the nitrogen atom to which they are bonded form a heterocyclic compound or a heteroaryl, possibly substituted.

In a particular embodiment, X represents a branched divalent radical complying with the formula —(CH₂)ₚ—CHRₓ—(CH₂)_{q} in which p and q are independently of each other an integer number ranging from 0 to 7, and the sum p+q is an integer number ranging from 1 to 7, and Rₓ is C₁₋₆alkyl, linear, branched or cyclic, C₁₋₆alkylC₁₋₆aryl; C₁₋₆alkyloxy; a side chain of a natural amino acid, or —OR^{G1}; —C(═O)^{G1}; where R^{G1} and R^{G2} are independently of each other a hydrogen atom or a C₁₋₆alkyl group, possibly substituted, or R^{G1} and R^{G2} conjointly with the nitrogen atoms to which they are bonded form a heterocyclic compound or a heteroaryl, possibly substituted.

According to a particular embodiment, A is —NY—, and the amino acid residue Z is bonded to the radical X via an amide bond, in particular complying with the formula Z′CONYX, in which the radical Z′CO— represents the amino acid residue Z, and X and Z are as defined above.

According to a particular embodiment, A is —CO—, and the amino acid residue Z is bonded to the radical X via a retro-inverso amide bond, in particular complying with the formula Z′NYCOX, in which the radical Z′NY— represents the amino acid residue Z, and X and Z are as defined above.

According to a particular embodiment, A is —O—, and the amino acid residue Z is bonded to the radical X via an ester bond, in particular complying with the formula Z′COOX, in which the radical Z′CO— represents the amino acid residue Z, and X and Z are as defined above.

According to a particular embodiment, A is —SO₂—, and the amino acid residue Z is bonded to the radical X via an sulfonamide bond, in particular complying with the formula Z′NYSO₂X, in which the radical Z′NY— represents the amino acid residue Z, and X and Z are as defined above.

According to a particular embodiment, A is —C(═S)—, and the amino acid residue Z is bonded to the radical X via an thioamide bond, in particular complying with the formula Z′NYC(S═O)X, in which the radical Z′NY— represents the amino acid residue Z, and X and Z are as defined above.

According to a particular embodiment, A is —S—, and the amino acid residue Z is bonded to the radical X via an thioester bond, in particular complying with the formula Z′C(═O)SX, in which the radical Z′C(═O)— represents the amino acid residue Z, and X and Z are as defined above.

According to a particular embodiment, the Y group, in the aforementioned formulae Z′CONYX, Z′NYCOX, Z′NYC(S═O)X and Z′NYSO₂X, represents a hydrogen atom or a protective group, in particular chosen from those described in the work “Protective Groups in Organic Synthesis” by T W Greene, P G M Wuts, Wiley-Interscience, New York, 4^{th} edition, 2007.

The compounds described herein can be substituted by substituents or chemical functions, which may be as numerous and varied as the chemical valency of the compound so permits. In general, the term “substituted”, preceded or not by the term “possibly”, and the substituents described in the formulae in the present document, designate the replacement of a hydrogen radical in a given structure with the radical of a specified substituent. When more than one position in a given structure can be substituted with more than one substituent selected from a specified group, the substituents may be the same or different at each position. The term “substituted” covers all the substituents of the organic compounds that are possible and can be envisaged by a person skilled in the art. According to one aspect, the substituents envisaged include any carbon substituent or heteroatom of organic compounds, whether they be cyclic or not, linear or branched, heterocyclic or carbocyclic, aromatic or not. With regard to the present invention, the heteroatoms, such as the nitrogen atom, can carry hydrogen atoms and/or any permissible substituent of organic compounds described in the present document, which satisfy the chemical valency of the said heteroatoms. In addition, the invention as described in the present document should under no circumstances be interpreted as being limited by the permissible substituents of the organic compounds. The combinations of substituents and chemical groups envisaged in the present invention are preferably those that result in the formation of stable compounds that can be used for the treatment and prevention of diseases, disorders and illnesses, such as those described in the present document. The substituents include, non-limitatively, alkyl; alkene; alkyne; cycloalkyl; cycloalkene; cycloalkyne; heteroalkyl; haloalkyl; aryl; heteroaryl; heterocyclic compound; alkylaryl; alkylheteroaryl; alkoxy; aryloxy; heteroalkoxy, heteroaryloxy; heteroalkythio; arylthio; heteroalkylthio; heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, heterocyclic compound, alkylaryl or alkylheteroaryl group in which the aryl, heteroaryl or heterocyclic radical is possibly substituted; or, when G represents —NR^{G2}—, R^{G1} and R^{G2} conjointly with the nitrogen atom to which they are bonded form a heterocyclic compound or a heteroaryl, possibly substituted.

Other examples of substituents generally acceptable for implementing the invention may appear to a person skilled in the art from reading the following examples, given by way of illustration.

The term “stable” preferably designates compounds that are sufficiently stable to allow their preparation, and the integrity of which is maintained for a sufficient period to allow their detection, and preferably for a sufficient period to be usable for the objectives detailed in the present document.

Halogen atom means an atom chosen from fluorine, bromine and iodine.

The alkyl radicals may comprise from 1 to 18 carbon atoms, in particular 1 to 12 carbon atoms, and especially 1 to 6 carbon atoms.

The alkene radicals make comprise 2 to 18 carbon atoms, and in particular 2 to 12 carbon atoms, and especially 2 to 6 carbon atoms. They may also comprise one or more double bonds.

The alkyne radicals make comprise 2 to 18 carbon atoms, and in particular 2 to 12 carbon atoms, and especially 2 to 6 carbon atoms. They may also comprise one or more triple bonds.

Unless mentioned to the contrary, the alkyl, alkene and alkyne radicals may be linear, branched or cyclic.

The term “heteroalkyl” designates an alkyl radical in which at least one carbon atom in the main chain has been replaced by a heteroatom. Thus a heteroalkyl designates an alkyl radical comprising, in its main chain, at least one heteroatom selected from nitrogen, sulphur, phosphorus, silicon, oxygen or selenium atoms in place of a carbon atom. Thus a C₁₋₆heteroalkyl radical designates a radical comprising 1 to 6 carbon atoms and at least one heteroatom selected from nitrogen, sulphur, phosphorus, silicon, oxygen or selenium atoms.

The term “aryl” designates a mono-, bi- or tricyclic hydrocarbon system comprising 1, 2 or 3 rings satisfying Hückel's aromaticity rule. For example, an aryl radical may be a phenyl, naphthyl, tetrahydronaphthyl, indanyl, indenyl and similar radical groups. The aryl radicals may comprise from 6 to 14 carbon atoms, and in particular 6 to 10 carbon atoms.

The term “heteroaryl” designates an unsaturated heterocyclic system comprising at least one aromatic ring, and 5 to 14 links, among which at least one link of the ring system is selected from S, O and N; 0, 1 or 2 links in the ring system are additional heteroatoms selected independently of each other from S, O and N; the rest of the links in the ring system being carbon atoms; the heteroaryl radical being bonded to the rest of the molecule via any one of the links in the ring system (whether it is a case of a carbon atom or a heteroatom). For example, a heteroaryl radical may be a pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl or isoquinolinyl radical, and similar radicals.

The arylalkyl and alkylaryl radicals may comprise 7 to 25 carbon atoms, in particular 7 to 20 carbon atoms, and especially 7 to 15 carbon atoms. In particular the arylalkyl radical may represent a benzyl.

The heteroarylalkyl and alkylheteroaryl radicals may comprise 7 to 25 carbon atoms, in particular 7 to 20 carbon atoms, and especially 7 to 15 carbon atoms.

When R2 and R3, R3 and R4 and/or R4 and R5 form together a ring or a heterocyclic compound, this may have 4 to 10 links, and in particular 6 to 8 links.

The term “heterocyclic compound” designates a saturated or unsaturated and non-aromatic mono- or polycyclic ring system comprising 5 to 20 links, and possibly comprising one or more rings with 5 to 6 links having between 1 and 3 heteroatoms selected independently of each other from S, O, N, P, Se and Si, in which (i) each ring with 5 links has 0 to 2 double bonds, and each link with 6 rings has 0 to 2 double bonds, (ii) the sulphur and/or nitrogen atoms are possibly oxidised, and (iii) the nitrogen atoms are possibly in the form of quaternary salts. For example, a heterocyclic radical may be a pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, or tetrahydrofuryl group.

A heterocyclic compound comprises in its ring system, apart from the carbon atoms, at least one heteroatom, in particular chosen from oxygen, nitrogen, sulfur, phosphorus, selenium and silicon.

The term “amine” or “amino” designates a radical complying with the formula —N(R)₂, in which each occurrence of R is independently of each other a hydrogen atom; an alkyl, heteroalkyl, alkene, alkyne, aryl, heteroaryl, arylalkyl, alkylaryl, heteroarylalkyl or alkyheteroaryl radical, possibly substituted; or in which the R groups form, with the nitrogen atom with which they are bonded, a heterocyclic compound or heteroaryl, possibly substituted. The amine function can possibly be in the form of a quaternary amine salt.

The term “carboxylic acid derivative” designates a radical complying with the formula —C(=0)R in which R is a hydrogen atom; a halogen; an alkyl, heteroalkyl, alkene, alkyne, aryl, heteroaryl, arylalkyl, alkylaryl, heteroarylalkyl or alkylheteroaryl radical, possibly substituted; or a -GR^{G1} function in which g is —O— or —NR^{G2}—, where R^{G2} is independently of R^{G1} a hydrogen atom; or an alkyl, heteroalkyl, alkene or alkyne function, linear, branched or cyclic, possibly substituted; or an aryl, heteroaryl, heterocycle, alkylaryl or alkylheteroaryl group in which the aryl, heteroaryl or heterocyclic radical is possibly substituted, or R^{G1} and R^{G2} conjointly with the nitrogen atom to which they are linked form a heterocyclic compound or a heteroaryl, possibly substituted.

The amino acid residues may be residues of natural amino acids or synthetic amino acids, in particular β, γ or ω amino acids. The amino acid residues may be in racemic form or in enantiomerically enriched form or even pure, in D or L form. The terminal amine or carboxyl functions (ie not linked to X or X′) and any lateral chemical functions of the amino acid residues may be protected or not.

In general terms all the α, β and γ amino acid residues, natural (L) or not (D, or synthetic), can be used. For example, a amino acids such as ornithine or norleucine, β amino acids such as β alanine, γ amino acids such as statine, or more exotic structures used in the synthesis of peptidomimetics such as for example TIC (1,2,3,4-tetrahydroisoquinoline-3-carboxylic acid), can be employed.

This does not constitute an exhaustive list.

The amino acid residues can for example be chosen from the group comprising:
natural amino acid residues, in particular glycine, alanine, valine, leucine, isoleucine, phenylalanine, tyrosine, tryptophan, glutamic acid, glutamine, aspartic acid, asparagine, serine, threonine, methionine, cysteine, lysine, arginine, histidine, proline,
“rare” amino acid residues, in particular hydroxyproline, hydroxylysine, allo-hydroxylysine, 6-N-methylysine, N-ethylglycine, N-methylglycine, N-ethylasparagine, allo-isoleucine, N-methylisoleucine, N-methylvaline, pyroglutamine, aminobutyric acid,
synthetic amino acid residues, in particular ornithine, norleucine, norvaline, cyclohexyl-alanine,
statine, and
TIC (1,2,3,4-tetrahydroisoquinoline-3-carboxylic acid).

These residues can be in racemic form or in D or L form.

The term “isolated”, when used to characterise the compounds of the invention, designates compounds that are (i) separated from at least one compound with which they are associated in nature, and/or (ii) produced, prepared or manufactured by human hand.

According to a first definition of the invention, the compounds comply with formula (I) as defined above, in which the following compounds are excluded:

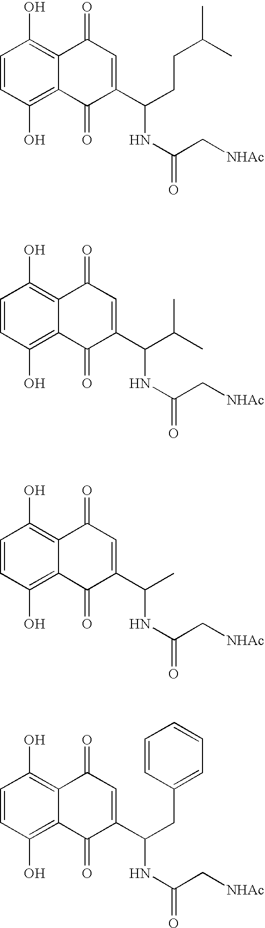

According to a second definition of the invention, the compounds comply with the following formula (i), or one of its pharmaceutically acceptable salts:

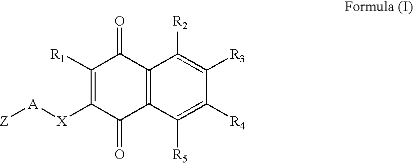

in which:
A is —O—, —S—, —SO₂—, —C(═S)—, —CO— or a chemical function such that Z′ and X′ are linked by a bioisosteric bond of the amine function
R1 represents a hydrogen atom; a halogen atom; a hydroxyl function, possibly substituted; an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; a nitro function; or an —X′-A′-Z′ function in which
   (i) X′ represents a divalent radical, in particular chosen from alkyls, alkenes or alkynes, linear, branched or cyclic, substituted or not, chiral or non-chiral, possibly interrupted by a heteroatom,
   (ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, in particular chosen from those described in the work “Protective Groups in Organic Synthesis” by T W Greene, P G M Wuts, Wiley-Interscience, New York, 4^{th} edition, 2007, and
   (iii) Z′ represents an amino acid residue, in particular D or L, natural or synthetic, in particular an α, β or γ amino acid residue, the terminal amine or carboxyl function (ie not linked to X′) and any lateral chemical functions of Z′ being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R2, R3, R4 and R5 represent independently of one another a hydrogen atom; a halogen atom; a hydroxyl function, possibly substituted, an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; or a nitro function;
   or R2 and R3, R3 and R4 and/or R4 and R5 form together a ring or a heterocyclic compound, possibly substituted,
X represents a divalent radical, in particular chosen from alkyls, alkenes or alkynes, linear, branched or cyclic, substituted or not, chiral or non-chiral, possibly interrupted by a heteroatom,
Z represents an amino acid residue, in particular D or L, natural or synthetic, in particular an α, β or γ amino acid residue, the terminal amine or carboxyl function (ie not linked to X) and any lateral chemical functions of Z being protected or not, where Z is linked to the X radical via a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z resulting from the reduction of the terminal carboxyl function of the Z aminoacid residue;
   or in which

### A is —NY—; and

(a)
R1 represents a hydrogen atom; a halogen atom; a hydroxyl function, possibly substituted, an alkyl, alkene, or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; a nitro function; or an —X′-A′-Z′ function in which;
   (i) X′ represents a divalent radical, in particular chosen from alkyls, alkenes or alkynes, linear, branched or cyclic, substituted or not, chiral or non-chiral, possibly interrupted by a heteroatom,
   (ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, in particular chosen from those described in the work “Protective Groups in Organic Synthesis” by T W Greene, P G M Wuts, Wiley-Interscience, New York, 4^{th} edition, 2007, and
   (iii) Z′ represents an amino acid residue, in particular D or L, natural or synthetic, in particular an α, β or γ amino acid residue, the terminal amine or carboxyl function (ie not linked to X′) and any lateral chemical functions of Z′ being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R3, R4 and R5 represent independently of one another a hydrogen atom; a halogen atom; a hydroxyl function, possibly substituted, an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; or a nitrofunction;
R2 represents, independently of R1, R2, R3, R4 and R5, a hydrogen atom; a halogen atom; a hydroxyl function, possibly substituted, an alkyl, alkene, or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups or a nitro function
   or R2 and R3, R3 and R4 and/or R4 and R5 form together a ring or a heterocyclic compound, possibly substituted,
X represents a divalent radical, in particular chosen from alkyls, alkenes or alkynes, linear, branched or cyclic, substituted or not, chiral or non-chiral, possibly interrupted by a heteroatom,
Y represents a hydrogen atom or a protective group, in particular chosen from those described in the work “Protective Groups in Organic Synthesis” by T W Greene, P. G. M. Wuts, Wiley-Interscience, New York, 4th edition, 2007, and
Z represents an amino acid residue, in particular D or L, natural or synthetic, in particular an α, β or γ amino acid residue, the terminal amine function (ie not linked to X) and any lateral chemical functions of Z being protected or not, where Z is linked to the X radical via an amide bond;
   (b)
R1 represents a hydrogen atom; a halogen atom; a hydroxyl function, possibly substituted, an alkyl, alkene, or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; a nitro function; or an —X′-A′-Z′ function in which;
   (i) X′ represents a divalent radical, in particular chosen from alkyls, alkenes or alkynes, linear, branched or cyclic, substituted or not, chiral or non-chiral, possibly interrupted by a heteroatom,
   (ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, in particular chosen from those described in the work “Protective Groups in Organic Synthesis” by T W Greene, P G M Wuts, Wiley-Interscience, New York, 4^{th} edition, 2007, and
   (iii) Z′ represents an amino acid residue, in particular D or L, natural or synthetic, in particular an α, β or γ amino acid residue, the terminal amine or carboxyl function (ie not linked to X′) and any lateral chemical functions of Z being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R3, R4 and R5 represent independently of one another a hydrogen atom; a halogen atom; a hydroxyl function, possibly substituted, an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; or a nitrofunction;
R2 represents a hydroxyl function; or R3 and R4 and/or R4 and R5 form together a ring or a heterocyclic compound, possibly substituted,
X represents a divalent radical, in particular chosen from alkyls, alkenes or alkynes, linear or cyclic, not substituted, possibly interrupted by a heteroatom,
Y represents a hydrogen atom or a protective group, in pan particular chosen from those described in the work “Protective Groups in Organic Synthesis” by T W Greene, P G M Wuts, Wiley-Interscience, New York, 4^{th} edition, 2007, and
Z represents an amino acid residue, in particular D or L, natural or synthetic, in particular an α, β or γ amino acid residue, the terminal amine function (ie not linked to X) and any lateral chemical functions of Z being protected or not, where Z is linked to the X radical via an amide bond;
   or in which:
   (c)
R1 represents a halogen atom; a hydroxyl function, possibly substituted, an alkyl, alkene, or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; a nitro function; or an —X′-A′-Z′ function in which;
   (i) X′ represents a divalent radical, in particular chosen from alkyls, alkenes or alkynes, linear, branched or cyclic, substituted or not, chiral or non-chiral, possibly interrupted by a heteroatom,
   (ii) A′ is —O—, —S—, —NY′—, —SO₂—, —C(═S)—, —CO— or a chemical function such that Z′ and X′. are linked by a bioisosteric bond of the amide function, where Y′ represents a hydrogen atom or a protective group, in particular chosen from those described in the work “Protective Groups in Organic Synthesis” by T W Greene, P G M Wuts, Wiley-Interscience, New York, 4^{th} edition, 2007, and
   (iii) Z′ represents an amino acid residue, in particular D or L, natural or synthetic, in particular an α, β or γ amino acid residue, the terminal amine or carboxyl function (ie not linked to X′) and any lateral chemical functions of Z being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R2 represents a hydroxyl function;
R3, R4 and R5 represent independently of one another a hydrogen atom; a halogen atom; a hydroxyl function, possibly substituted; an alkyl, alkene or alkyne radical, comprising from 1 or 2 to 18 carbon atoms, possibly substituted, in particular by one or more amino, carboxylic acid, carboxylic acid derivative, alkoxy, aryl or hydroxy groups; an aryl radical, possibly substituted, in particular by one or more amino, carboxylic acid, carboxylic acid derivative, alkoxy, aryl or hydroxy groups; an arylalkyl or alkylaryl radical, possibly substituted, in particular by one or more amino, carboxylic acid, carboxylic acid derivative, alkoxy, aryl or hydroxy groups; or a nitro function;
   or R3 and R4 and/or R4 and R5 form together a ring or a heterocyclic compound, possibly substituted,
X represents a divalent radical, in particular chosen from alkyls, alkenes or alkynes, linear, branched or cyclic, substituted or not, possibly interrupted by a heteroatom,
Y represents a hydrogen atom or a protective group, and
Z represents an amino acid residue, in particular D or L, natural or synthetic, in particular an α, β or γ amino acid residue, the terminal amine or carboxyl (ie not linked to X) and any lateral chemical functions of Z being protected or not, where Z is linked to the X radical via an amide bond,
   in particular, for the compounds described in parts (a), (b) and (c) above, and for the compounds of formula (I) above where A is —O—, —S—, —SO₂—, —C(═S)—, —CO— or a chemical function such that Z′ and X′ are linked by a bioisosteric bond of the amide function, X and X′ are, independently of each other, possibly substituted by one or more chemical functions such as a lateral chain of a natural amino acid; C₁₋₆alkyl; C₂₋₆alkene; C₂₋₆alkyne; C₃₋₈cycloalkyl; C₁₋₆heteroalkyl; C₁₋₆haloalkyl; C₆₋₁₀aryl; C₃₋₁₀heteroaryl; C₅₋₂₀heterocyclic; C₁₋₆alkylC₆₋₁₀aryl; C₁₋₆alkylC₃₋₁₀heteroaryl; C₁₋₆alkoxy; C₆₋₁₀aryloxy; C₃₋₈heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}, —C(═O)—, —S(═O)—, —SO₂—, —C(═O)O—, —C(═O)NR^{G2}—, —C(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G2})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, heterocyclic compound, alkylaryl or alkylheteroaryl group in which the aryl, heteroaryl or heterocyclic radical is possibly substituted; or, when G represents —NR^{G2}—, R^{G1} and R^{G2} conjointly with the nitrogen atom to which they are bonded form a heterocyclic compound or a heteroaryl, possibly substituted,

According to a particular embodiment, the compounds comply with one of the following formulae:

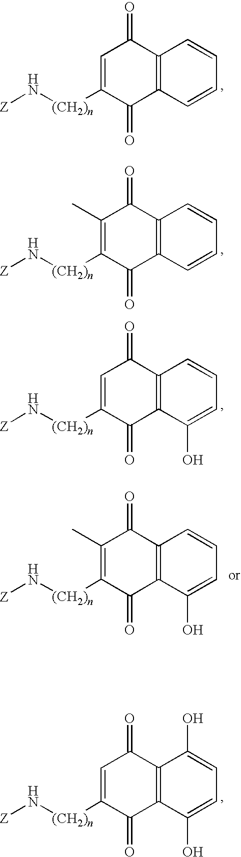

in which n is an integer number ranging from 1 to 12, in particular from 2 to 8, and Z represents an amino acid residue, in particular D or L, natural or synthetic, in particular an α, β or γ amino acid residue, the terminal amine function (ie not linked to —NH—) and any lateral chemical functions of Z being protected or not, where Z is linked to the —NH— radical via an amide bond.

According to a particular embodiment, the compounds comply with one of the following formulae:

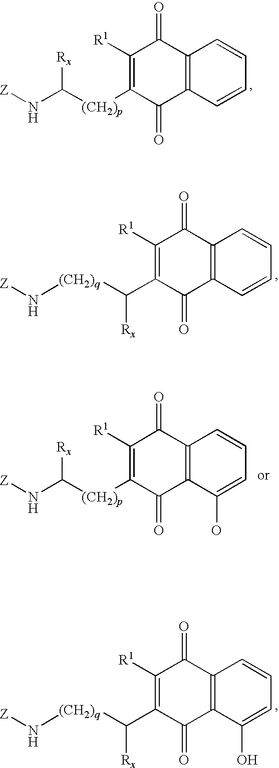

in which Z is as defined above; p and q are independently of each other integers ranging from 0 to 12, in particular from 0 to 7; R1 is as defined previously; and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1} function in which G is —O—, —S—, —NR^{G2}, —C(═O)—, —S(═O)—, —SO₂—, —C(═O)O—, —C(═O)NR^{G2}—, —OC(═O), —NR^{G2}C(═O)—, —OC(═O)0-, —OC(═O)NR^{G2}—, —NR^{G2}C(═O)—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—NR^{G2}C(═NR^{G3})—, —NR^{G2}SO₂—, —NR^{G2} SO₂NR^{G2}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, heterocyclic compound, alkylaryl or alkylheteroaryl group in which the aryl, heteroaryl or heterocyclic radical is possibly substituted.

According to a particular embodiment, the compounds comply with one of the following formulae:

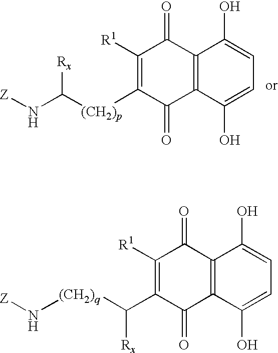

in which Z is as defined above; p and q are independently of each other integers ranging from 0 to 12, in particular from 0 to 7; except when R1 is a hydrogen atom, in which case p and q are independently of each other in integers ranging from 1 to 12, in particular from 1 to 7; R1 is as defined previously; and R1 is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₈heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2} SO₂—, —NR^{G2}SO₂NR^{G3}—, —NR^{G2} (═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, alkylaryl or alkylheteroaryl group in which the aryl or heteroaryl is possibly substituted.

According to a particular embodiment, the compounds comply with one of the following formulae:

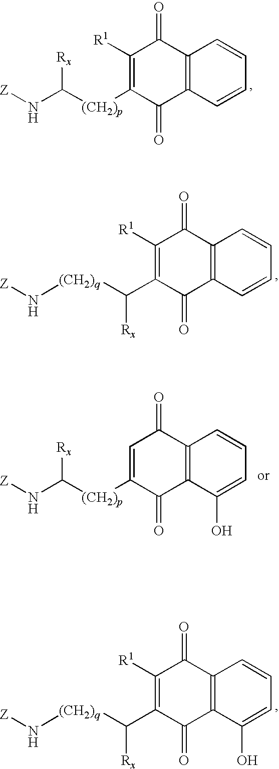

in which Z is as defined above; p and q are independently of each other integers ranging from 0 to 12, in particular from 0 to 7, and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₈heteroaryl; C₁₋₆alkoxy; C₆₋₁₀aryloxy; C₃₋₈heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which g is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2} SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—C(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, alkylaryl or alkylheteroaryl group in which the aryl or heteroaryl radical is possibly substituted.

According to a particular embodiment, the compounds comply with one of the following formulae:

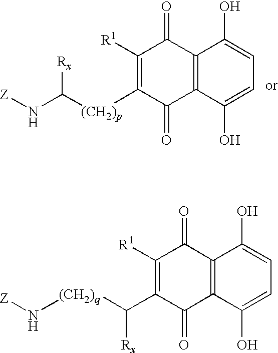

in which Z is as defined above; p and q are independently of each other integers ranging from 1 to 12, in particular from 1 to 7, and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1}-function in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2}SO₂—, —NR^{G2} SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, alkylaryl or alkylheteroaryl group in which the aryl or heteroaryl radical is possibly substituted.

According to a particular embodiment, the compounds comply with one of the following formulae:

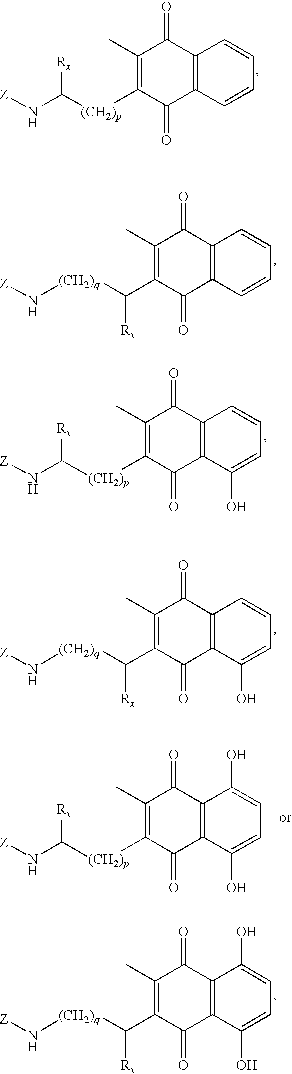

in which Z is defined above; p and q are independently of each other integers ranging from 0 to 12, in particular from 0 to 7, and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—, —NR^{G2}C(═NR^{G3})—, —NR^{G2} SO₂—, —NR^{G2} SO₂NR^{G3}—, —NR^{G2}C(═S)—, —SC(═S)NR^{G2}—, —NR^{G2}C(═S)S—, —NR^{G2}C(═S)NR^{G2}—, —SC(═N—C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or an alkyl, heteroalkyl, alkene or alkyne function, linear, branched or cyclic, possibly substituted; or an aryl, heteroaryl, alkylaryl or alkylheteroaryl group in which the aryl or heteroaryl radical is possibly substituted.

According to a particular embodiment, the compounds comply with one of the following formulae:

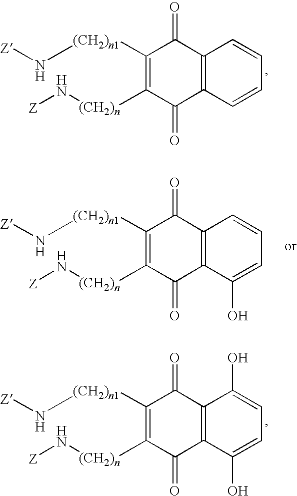

in which n and n1 are independently of each other integers ranging from 1 to 12, in particular from 1 to 5; and Z and Z′ are independently of each other an amino acid residue, in particular D or L, natural or synthetic, in particular an α, β or γ amino acid residue, the terminal amine function (ie not linked to —NH—) and any lateral chemical functions of Z/Z′ being protected or not, where Z and Z′ are linked to the radical —NH— via an amide bond.

More particularly, the compounds according to the invention can comply with the following formula (II) or with one of its pharmaceutically acceptable salts:

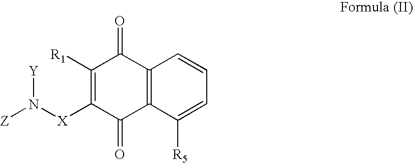

in which
—R1 represents a hydrogen atom, an alkyl radical comprising 1 to 6 carbon atoms, or a —(CH₂)ₙ₁—NY′-Z′ group, where
n1 represents an integer number ranging from 1 to 12, in particular from 1 to 6, more especially from 1 to 5, and in particular 1 to 2, and
Y and Y′ represent independently of each other a hydrogen atom or a protective group, and
Z and Z′ represent independently of each other an amino acid residue, in particular D or L, natural or synthetic, in particular an α, β or γ amino acid residue, the terminal amine function (ie not linked to —NY— or —NY′—) and any lateral chemical functions of Z and Z′ being protected or not, where Z and Z′ are linked to the —NY— or —NH′— radical, respectively, via an amide bond;
X represents a —(CH₂)ₙ— group, n represents an integer number ranging from 1 to 12, in particular 1 to 6, more particularly from 1 to 5, and especially from 1 to 2, and
R5 represents a hydrogen atom, a halogen atom or a hydroxyl function, possible substituted.

According to a first embodiment, the compound according to the invention complies with formula (II), in which R1 represents an alkyl radical comprising from 1 to 6 carbon atoms, in particular from 1 to 4 carbon atoms, especially from 1 to 2 carbon atoms, or even is the methyl radical.

According to a first variant, these compounds belong to the family of Plumbagones, and then comply with formula (II) in which R1 represents a methyl radical and R5 represents a hydroxyl function.

According to a second variant, these compounds belong to the family of Menadiones, and then comply with formula (II) in which R1 represents a methyl radical and R5 represents a hydrogen atom.

According to a second embodiment, the compound according to the invention complies with formula (II) in which R1 represents a hydrogen atom.

More especially these compounds belong to the family of Juglones with single substitution, when they comply with formula (II) in which R1 represents a hydrogen atom and R5 represents a hydroxyl function.

According to a third embodiment, the compound according to the invention complies with formula (II) in which R1 represents a —(CH₂)ₙ₁—NY′—COCHRNH₂ group, where
COCHRNH₂ represents a natural or synthetic amino acid residue, D or L, in which R designates the lateral chain of the said amino acid residue,
n1 represents an integer number ranging from 1 to 5, and in particular from 1 to 2, and
Y′ represents a hydrogen atom or an protective group, in particular chosen from those described in the work “Protective Groups in Organic Synthesis” by T W Green, P g M Wuts, Wiley-Interscience, New York, 4^{th} edition 2007.

In particular, these compounds belong to the family of Juglones with double substitution when they comply with formula (II) in which R1 represents a —(CH₂)ₙ₁—NY′—COCHRNE₂ group, and R5 represents a hydroxyl function.

The compounds of formula (I) or (II) can where necessary be in solvated form, in salt form, or other physiologically acceptable derivatives. The salts and solvents that are acceptable for pharmaceutical use are generally those in which the counter ion or associated solvent is pharmaceutically acceptable.

The salts that can be used may be acids or organic or mineral bases. Among the acceptable salts of addition acids, those formed from hydrochloric, hydrobromic, sulphuric, citric, tartric, phosphoric, lactic, pyruvic, acetic, trifluoracetic, phenylacetic or triphenylacetic acid can be cited.

There can also be cited, among the acceptable basic salts, the salts of alkali metals, such as sodium or potassium, the salts of alkaline-earth metals, such as calcium and magnesium, and the salts formed from organic bases, such as mono-, di- or tri-substituted amines.

According to another of its aspects, an object of the invention is a pharmaceutical composition comprising, by way of active agent, at least one compound as defined above in a pharmaceutically acceptable carrier.

In particular the composition may be anti-cancerous and/or pro-apoptotic and/or anti-proliferative.

In the pharmaceutical composition, the compounds are used in an effective quantity. This will be determined by a person skilled in the art, according to various parameters, in particular with respect to the substance used, and the age, weight and physical state of the patient, the administration mode, and the regime required. The person skilled in the art will be in a position to determine the administration mode and the dosage for each patient.

In particular, the compound according to the invention can be administered at a dose ranging from 0.1 to 5000 mg per day and per patient.

The pharmaceutical composition may comprise a quantity of compound according to the invention ranging from 0.1 mg to 5 g.

The pharmaceutical composition may be administered in any form, topical or systemic, in particular in parenteral or enteral form.

When the composition or medication are administered by enteral route, it may be in the form of tablets, capsules, pills, syrups, suspensions, solutions, powder, granules, emulsions or microspheres.

In the case of administration by parenteral route, the composition may be in the form of solutions or suspensions for perfusion or injection.

The composition may also comprise at least one additive, chosen in particular from dyes, flavourings and preservatives. Naturally, a person skilled in the art will ensure that he chooses the additive or additives so that the advantageous properties intrinsically attached to the invention are not, or substantially not, impaired by the envisaged addition.

According to a particular embodiment the composition according to the invention can also comprise another compound intended to treat cancer. Among the compounds that can be used according to the invention, doxorubicine, the trade name of which is Adriamycine®, epothilone, paclitaxel, the trade name of which is Taxol®, and cis-platine can be cited.

According to yet another of its aspects, an object of the invention is the use of at least one compound as defined above for the preparation of a pharmaceutical composition intended to treat and/or prevent an abnormal proliferation of cells.

The said composition can be intended for human and/or veterinary medicine, and in particular it can be intended to treat or prevent at least one cancer chosen from pancreatic cancer, cancers of the oropharynx, stomach cancer, cancer of the oesophagus, colon and rectal cancer, brain tumours, in particular gliomers, ovarian cancer, liver cancer, kidney cancer, cancer of the larynx, thyroid cancer, lung cancer, bone cancer, multiple myelomas, mesotheliomas and melanomas, skin cancer, breast cancer, prostate cancer, bladder cancer, cancer of the uterus, testicular cancer, non-Hodgkin's lymphoma, leukaemia, Hodgkin's disease and soft-tissue cancers, as well as secondary locations metastatic of the aforementioned cancers.

“Abnormal proliferation” means a proliferation that is independent of the normal regulation mechanisms, for example the stoppage of cell proliferation due to the involvement of apoptosis (programmed cell death).

According to another of its aspects, another object of the invention is a pro-apoptotic and/or anti-proliferative composition comprising at least one compound as defined above.

According to yet another of its aspects, an object of the invention is the use of at least one compound as defined above as a pro-apoptotic and/or anti-proliferative agent.

The following examples are given by way of indication and without any character limitative of the invention.

Advantages other than those described in the present application may also appear to a person skilled in the art from reading the following examples given by way of illustration.

### EXAMPLES

In the examples, the following abbreviations are used.

AcOEt=ethyl acetate
AgNO₃=silver nitrate
Boc₂O=di-tert-butyl dicarbonate
CH₂Cl₂=dichloromethane
CH₃CN=acetonitrile
DCHA=dicyclohexylamine
DIEA=diisopropylethylamine
H₂O=water
HBTU=o-benzotriazolyl-N,N,N,N′-tetramethyluronium hexafluorophosphate

### HOBt=N-hydroxybenzotriazole

MgSO₄=magnesium sulphate
ml=millilitre
mmol=millimoles
NaCl=sodium chloride
NaOH=sodium hydroxide
(NH₄)₂S₂O₈=ammonium persulfate
TFA=trifluororacetic acid

The protected amino acids come from Novabiochem, in particular Boc-L-Ala-OH, Boc-L-Ser(OtBu)—OH, Boc-L-Thr(OtBu)—OH, Boc-D-Val-OH, Boc-D-Trp-OH, Boc-L-Thr(OBn)—OH and Boc-D-Tyr(OBn)—OH.

All the other chemical compounds used come from Aldrich, Fluka or Acros and are of standard quality.

The solvents are distilled before use.

### Example 1

### Synthesis of N-tert-butyloxycarbonyl-2-aminoacetic acid (Boc-N-glycine)

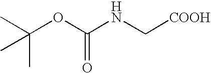

In a 500 ml flask, 4.002 g of glycine (1 eq.; 53 mmol) is introduced, 150 ml of a mixture of dioxane and water (ratio 2:1) is poured in, that is to say 100 ml of dioxane and 50 ml of water, and then an aqueous solution is added, prepared form 2 g of NaOH for 50 ml of water. Cooling is carried out by means of an ice bath and 12.72 g of di-tert-butyl dicarbonate is added (1.1 eq.; 58 mmol). The reaction medium is stirred throughout the night at room temperature. The dioxane and water are evaporated and then 40 ml of ethyl acetate is added, and washing is carried out by means of 5% citric acid and then saturated sodium chloride, and an extraction of the aqueous phases is carried with dichloromethane. The organic phases are collected together, and dried on MgSO₄, and evaporated. 7.705 g of a white solid (yield=83%) is obtained. The product obtained does not require any additional purification before use thereof.

### Example 2

### Synthesis of N-tert-butyloxycarbonyl-4-aminobutanoic acid

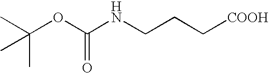

In a 250 ml flask, containing 3.004 g of 4-aminobutanoic acid (1 eq.; 29 mmol), 75 ml of a mixture of dioxane and water (ratio 2:1) is added, that is to say 50 ml of dioxane and 25 ml of water. A dilute solution of a solution of NaOH (1 g of NaOH for 25 ml of water) is added. The flask is placed in an ice bath and 6.984 g of Boc₂O is added (di-tert-butyl dicarbonate 1.1 eq.; 31 mmol), then the mixture is left under stirring for the whole night at room temperature. Once the reaction is ended, the dioxane and water are evaporated and the reaction medium is taken up with 30 ml of AcOEt, washing is carried out with 5% aqueous citric acid (twice 10 ml), and then with saturated NaCl. The organic phases are re-extracted with CH₂Cl₂, brought together and then dried on MgSO₄ and evaporated. 5.182 g of a colourless viscous oil (yield=88%) is obtained. The product obtained does not require any additional purification after use thereof.

### Example 3

### Synthesis of 2-methyl-3-(N′-tert-butyloxycarbonyl-aminomethyl)-1,4-naphthoquinone

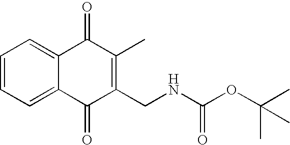

In a 25 ml flask containing 1200 g of commercial menadione (Aldrich Co 1 eq.; 6.9 mmol), 3.660 g of the compound of example 1 (3 eq.; 20 mmol) is introduced, and 0.355 g of silver nitrate (0.3 eq.; 2.07 mmol), a mixture of acetonitrile and water (ratio 7:3) is added, that is to say 12 ml of CH₃CN and 6 ml of H₂O, the temperature of the mixture is raised to 65° C. In an addition bulb, 0.822 g of ammonium peroxodisulfate (1.3 eq.; 3.6 mmol) dissolved in 7 ml of CH₃CN:H₂O mixture (ratio 7:3) is prepared. The addition is carried out over two hours, the mixture is maintained under stirring for one hour more at 65° C. Next extraction is carried three times with CH₂Cl₂, the organic phases are washed once with water and then are dried on MgSO₄ and evaporated. The product is then purified by column chromatography with 10% AcOEt:90% cyclohexane as eluant. 0.961 g of an orange solid (38%) is obtained. For more information the article by Anderson J M and Kochi J K in J Am Chem Soc 1970, 92(6), 1651-1659 can be consulted.

### Example 4

### Synthesis of 2-methyl-3-(N′-tert-butyloxycarbonyl-2-aminoethyl)-1,4-naphthoquinone

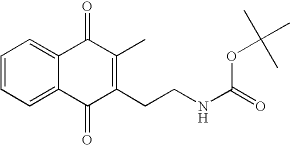

In a 50 ml flask, 0.500 g of menadione (1 eq.; 2.9 mmol), 1.638 g (3 eq.; 8.71 mmol) of BocβAla (Aldrich Co) and 0.147 g of AgNO₃ (0.3 eq.; 0.447 mmol) are weighed. The acetonitrile and water mixture (ratio 7:3) is added, that is to say 5.0 ml of CH₃CN and 2.5 ml of H₂O, and the temperature of the mixture is raised to 65° C. The addition of 0.861 g of (NH₄)S₂O₈ (1.3 eq.; 1.93 mmol) dissolved in 3 ml of a mixture of CH₃CN and H₂O takes place over two hours. The reaction medium is then maintained under stirring for one hour at 65° C. and then extraction is carried out with CH₂Cl₂ three times and the etherated phases are washed just once with H₂O. The organic phases are dried on MgSO₄ and evaporated. The product is purified by column chromatography with 10% AcOEt: 90% cyclohexane as an eluant: and 1.420 g of an orange oil (yield=46%) is obtained.

### Example 5

### Synthesis of 2-methyl-3-(methyl ammonium)-1,4-naphthoquinione trifluoracetate

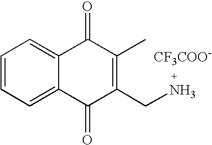

In a 25 ml flask immersed in an ice bath 1.277 g (1 eq.; 4.23 mmol) of the compound of example 3 is dissolved in a mixture of trifluoracetic acid and dichloromethane (ratio 1:1), that is to say 8.5 ml of each solvent. The reaction medium is stirred for six hours under nitrogen and at room temperature. The TFA and CH₂Cl₂ are then evaporated and taken up in toluene in order to remove the excess TFA. The flask is placed under vacuum for two days and 1.300 g of a brown solid (yield=97%) is obtained.

### Example 6

### Synthesis of 2-methyl-3-(ethyl ammonium)-1,4-naphthoquinone trifluoracetate

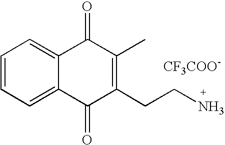

According to the operating protocol previously described for the compound of example 5, in a 25 ml flask, 0.881 g of the compound of example 1 is introduced (1 eq.: 2.79 mmol), and 5.5 ml of CH₂Cl₂ and 5.5 ml of TFA are added (in an ice bath). During evaporation the medium is taken up several times in toluene and 0.916 g of a brown solid is recovered (yield=99%).

### Example 7

### Syntheses of 2-methyl-3-(N′-tert-butyloxycarbonyl-L-alaninyl-aminomethyl)-1,4-naphthoquinone

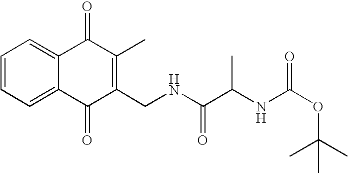

In a 10 ml flask, 0.037 g of BocAlaOH (1.2 eq.; 0.388 mmol), 0.183 g of HBTU (1.5 eq.; 0.484 mmol) and 0.052 g of HOBt (1.2 eq.; 0.388 mmol) are weighed. 3 ml of distilled CH₂Cl₂ is added, as well as 0.339 ml of DIEA (5 eq.; 1.94 mmol). The reaction medium is stirred under nitrogen for two hours in order to activate the acid. Next 0.102 g (1 eq.; 0.323 mmol) of the compound of example 5 is added, and then is left under stirring for two hours. The CH₂Cl₂ is evaporated, the medium is taken up in 30 ml of AcOEt and three washings are carried out with water. The organic phase is dried on MgSO₄ and evaporated. The product is then purified on a chromatography column with 40% AcOEt:60% Cyclo as an eluant and 0.030 g of a yellow solid is obtained (yield=25%).

### Example 8

### Synthesis of 2-methyl-3-(N′-tert-butyloxycarbonyl-L-serinyl(OtBu)-aminomethyl)-1,4-naphthoquinone

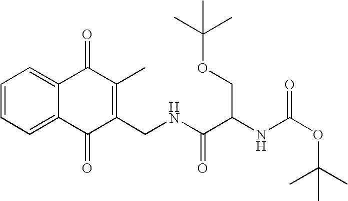

Firstly, before carrying out the coupling, it is necessary to deprotect the carboxylic acid function of the commercial serine. For this purpose there is introduced into a small decanting bulb 0.170 g of BocSer(tBu)OH.DCHA (0.38 mmol), to which there are added 3 ml of AcOEt, 3 ml of water and 0.4 ml of H₂SO₄ (2M). The aqueous phase is extracted with AcOEt once and the organic phase is washed three times with water. Evaporation is carried out and 0.077 g of a colourless oil is obtained, which is the serine residue in carboxylic acid form. This oil (1.2 eq.; 0.294 mmol) has added to it, as described previously for the compound of example 7, 0.139 g of HBTU (1.5 eq.; 0.368 mmol), 0.039 g of HOBt (1.2 eq.; 0.294 mmol), 3 ml of CH₂Cl₂ and 0.213 ml of DIEA. After two hours of activation, next 0.102 g of compound (5) (1 eq.; 0.323 mmol) is added and the whole is left under stirring for two hours. After washing with water, the product is purified on a chromatography column with 30% AcOEt; 70% Cyclo, and 0.080 g of an orange oil is obtained (yield=62%).

### Example 9

### Synthesis of 2-methyl-3-(N′-tert-butyloxycarbonyl-D-valinyl-aminomethyl)-1,4-naphthoquinone

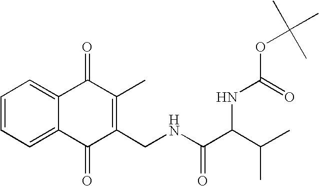

According to the coupling procedure described for the compound of example 7, 0.084 g of BocDValOH (1.2 eq.; 0.376 mmol), 0.183 g of HBTU (1.5 eq.; 0.484 mmol), 0.052 g of HOBt (1.2 eq.; 0.376 mmol), 3 ml of CH₂Cl₂ and 0.339 ml of DIEA are used. The activation is left to act for two hours under nitrogen. Next 0.100 g of the compound of example 5 is added and, after washing and purification of the product with 40% AcOEt; 60% Cyclo, 0.080 g of an orange oil is obtained (yield=62%).

### Example 10

### Synthesis of 2-methyl-3-(N′-tert-butyloxycarbonyl-L-threoninyl(OtBu)-aminomethyl)-1,4-naphthoquinone

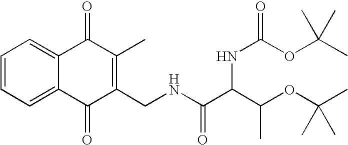

According to the coupling procedure described for the compound of example 7, 0.106 g of BocThr(tBu)OH (1.2 eq.; 0.384 mmol), 0.183 g of HBTU (1.5 eq.; 0.482 mmol), 0.052 of HOBt (1.2 eq; 0.388 mmol), 3 ml of CH₂Cl₂, and 0.338 ml of DIEA are used. Stirring is carried out for two hours under nitrogen and then 0.100 g of the compound of example 5 is added and after washing and purification carried out with 40% AcOEt; 60% Cyclo, 0.127 g of an orange oil is obtained (yield=86%).

### Example 11

### Synthesis of 2-methyl-3-(N′-tert-butyloxycarbonyl-glycinyl-aminomethyl)-1,4-naphthoquinone

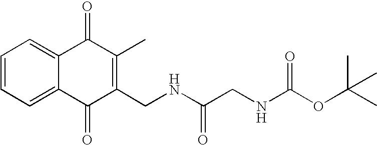

According to the coupling procedure described for the compound of example 7, 0.066 g of BocGlyOH (1.2 eq.; 0.0308 mmol), 0.183 g of HBTU (1.5 eq.; 0.485 mmol) 0.052 g of HOBt (1.2 eq.; 0.388 mmol), 3 ml of CH₂Cl₂ and 0.338 ml of DIEA are used. Stirring is carried out for two hours under nitrogen and then 0.100 g of the compound of example 5 is introduced and, after purification with 50% AcOEt; 50% Cyclo, 0.080 g of an orange oil is obtained (yield=71%).

### Example 12

### Synthesis of 2-methyl-3-(N′-tert-butyloxycarbonyl-L-alaninyl-aminoethyl)-1,4-naphthoquinone

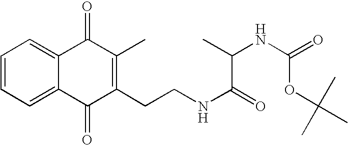

According to the coupling method described for the compound of example 7, in a 10 ml flask, 0.035 g of BocAlaOH (1.2 eq.; 0.18 mmol), 0.090 g of HBTU (1.5 eq.; 0.22 mmol), and 0.025 g of HOBt (1.2 eq.; 0.18 mmol) are weighed. 3 ml of distilled CH₂Cl₂ is added, along with 0.135 ml of DIEA (5 eq.; 0.76 mmol). The reaction medium is stirred under nitrogen for two hours in order to activate the acid. Next 0.05 g (1 eq.; 0.15 mmol) of the compound of example 6 is added and left under stirring for two hours. CH₂Cl₂ is evaporated and the medium is put in 30 ml of AcOEt, and three washings are carried out with water. The organic phase is dried on MgSO₄ and evaporation is carried out. The product is then purified on a chromatography column with 50% AcOEt: 50% Cyclo as an eluant and 0.030 g of a yellow oil is obtained (yield=52%).

### Example 13

### Synthesis of 2-methyl-3-(N′-tert-butyloxycarbonyl-L-serinyl(OtBu)-aminoethyl)-1,4-naphthoquinone

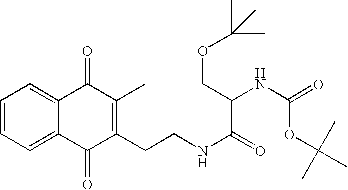

As with the compound of example 8, the DCHA salt permitting crystallisation of the serine residue must the hydrolysed: for this, in a small decanting bulb, 0.075 g of BocSer(tBu)OH.DCHA, that is to say (0.17 mmol) is introduced, to which 2 ml of AcOEt, 2 ml of water and 0.2 ml of H₂SO₄ (2M) is added. The aqueous phase is extracted with AcOEt once and the organic phase is washed three times with water. Evaporation is carried out and 0.045 g of a colourless oil, which is the serine residue in carboxylic acid form, is obtained. This oil (1.2 eq.; 0.17 mmol) is added as described previously for the compound of example 7 with 0.087 g of HBTU (1.5 eq.; 0.23 mmol), 0.020 g of HOBt (1.2 eq.; 0.15 mmol), 3 ml of CH₂Cl₂ and 0.135 ml of DIEA. After two hours of activation, next 0.050 g of the compound of example 6 (1 eq.; 0.15 mmol) is added and the whole is left under stirring for two hours. After washing with water, the product is purified on a chromatography column with 30% AcOEt; 70% Cyclo, and 0.040 g of an orange solid is obtained (yield=58%).

### Example 14

### Synthesis of 2-methyl-3-(N′-tert-butyloxycarbonyl-D-valinyl-aminoethyl)-1,4-naphthoquinone

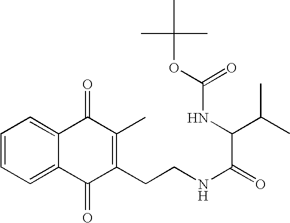

According to the coupling procedure described for the compound of example 7, 0.037 g of Boc-D-ValOH (1.2 eq; 0.17 mmol), 0.087 g of HBTU (1.5 eq.; 0.23 mmol), 0.020 of HOBt (1.2 eq; 0.15 mmol), 3 ml of CH₂Cl₂ and 0.135 ml of DIEA is used. The activation is left to act for two hours under nitrogen. Next 0.050 g of the compound of example 6 is added and, after washing and purification of the product on a chromatography column with 25% AcOEt; 75% Cyclo, 0.034 g of a yellow solid is obtained (yield=55%).

### Example 15

### Synthesis of 2-methyl-3-(N′-tert-butyloxycarbonyl-L-threoninyl(OtBu)-1,4-naphthoquinone

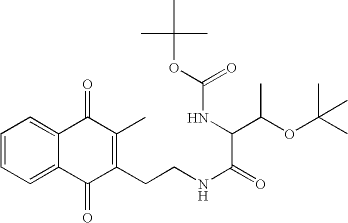

According to the coupling procedure described for the compound of example 7, 0.42 g of BocThr(tBu)OH (1.2 eq.; 0.17 mmol), 0.087 g of HBTU (1.5 eq.; 0.23 mmol), 0.020 g of HOBt (1.2 eq.; 0.15 mmol), 3 ml of CH₂Cl₂, and 0.135 ml of DIEA are used. The whole is stirred for two hours under nitrogen and then 0.050 g of the product of example 6 is added and, after washing and purification carried out with 20% AcOEt; 80% Cyclo, 0.043 g of a yellow oil is obtained (yield=61%).

### Example 16

### Synthesis of 2-methyl-3-(N′-tert-butyloxycarbonyl-glycinyl-aminoethyl)-1,4-naphthoquinone

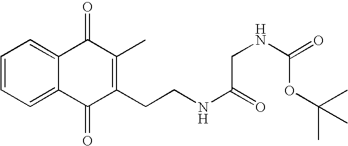

According to the coupling procedure described for the compound of example 7, 0.040 g of BocGlyOH (1.5 eq.; 0.23 mmol), 0.098 g of HBTU (1.5 eq; 0.23 mmol) 0.021 g of HOBt (1.2 eq.; 0.15 mmol), 3 ml of CH₂Cl₂, and 0.135 ml of DIEA are used. The whole is stirred for two hours under nitrogen and then 0.050 g of the compound of example 6 is introduced and, after purification with 40% AcOEt; 60% Cyclo, 0.035 g of an orange oil is obtained (yield=63%).

### Example 17

### Synthesis of 2-methyl-3-(L-alaninyl-aminomethyl)-1,4-naphthoquinone chlorhydrate

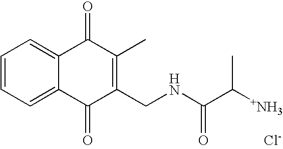

According to the operating protocol described previously for the compound of example 5, in a 10 ml flask 0.030 g of the compound of example 7 (1 eq.; 0.080 mmol), is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.022 g of a yellow-orange solid is recovered (yield=89%).

### Example 18

### Synthesis of 2-methyl-3-(L-serinyl-aminomethyl)-1,4-naphthoquinone chlorhydrate

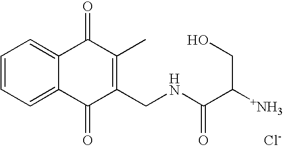

According to the operating protocol described previously for the compound of example 5, in a 10 ml flask 0.030 g of the compound of example 8 (1 eq.; 0.067 mmol) is introduced, and 2 ml of CH₂Cl₂ and 2 ml of TFA are added (in an ice bath). During evaporation the medium is taken up in toluene several times, and then the brown solid obtained is taken in a solution of 1M HCl in Et₂O for 30 minutes. The solvent is evaporated and the operation is repeated with the hydrochloric acid solution in ether twice. After evaporation and drying, 0.020 g of an orange solid is recovered (yield=91%).

### Example 19

### Synthesis of 2-methyl-3-(D-valinyl-aminomethyl)-1,4-naphthoquinone chlorhydrate

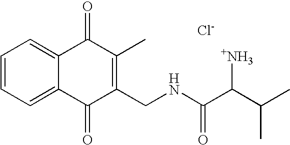

According to the operating protocol described previously for the compound of example 5, in a 10 ml flask 0.030 g of the compound of example 9 (1 eq.; 0.075 mmol) is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.023 g of a yellow solid is recovered (yield=91%).

### Example 20

### Synthesis of 2-methyl-3-(L-threoninyl-aminomethyl)-1,4-naphthoquinone chlorohydrate

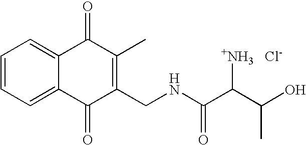

According to the operating protocol described previously for the compound of example 5, in a 10 ml flask 0.030 g of the compound of example 10 (1 eq.; 0.065 mmol) is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.021 g of an orange-brown solid is recovered (yield=95%).

### Example 21

### Synthesis of 2-methyl-3-(glycinyl-aminomethyl)-1,4-naphthoquinone chlorohydrate

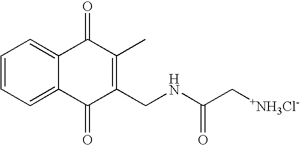

According to the operating protocol described for the compound of example 5, in a 10 ml flask 0.030 g of the compound of example 11 (1 eq.; 0.084 mmol), is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.022 g of an orange-brown solid is recovered (yield=89%).

### Example 22

### Synthesis of 2-methyl-3-(L-alaninyl-aminoethyl)-1,4-naphthoquinone chlorohydrate

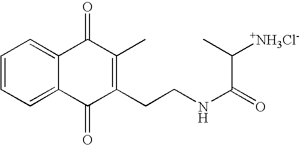

According to the operating protocol described for the compound of example 5, in a 10 ml flask 0.030 g of the compound of example 12 (1 eq.; 0.077 mmol) is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.023 g of an orange-brown solid is recovered (yield=92%).

### Example 23

### Synthesis of 2-methyl-3-(L-serinyl-aminoethyl)-1,4-naphthoquinone chlorohydrate

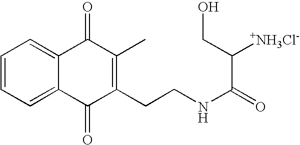

According to the operating protocol described for the compound of example 5, in a 10 ml flask 0.030 g of the compound of example 13 (1 eq.; 0.077 mmol), is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.023 g of an orange-brown solid is recovered (yield=92%).

### Example 24

### Synthesis of 2-methyl-3-(D-valinyl-aminoethyl)-1,4-naphthoquinone chlorohydrate

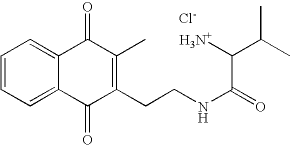

According to the operating protocol described for the compound of example 5, in a 10 ml flask 0.030 g of the compound of example 14 (1 eq.; 0.077 mmol) is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. With evaporation and drying, 0.023 g of a brown solid is recovered (yield=91%).

### Example 25

### Synthesis of 2-methyl-3-(L-threoninyl-methyl)-1,4-naphthoquinone chlorohydrate

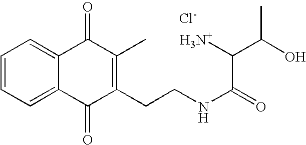

According to the operating protocol described for the compound of example 5, in a 10 ml flask 0.030 g of the compound of example 15 (1 eq.; 0.077 mmol) is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.020 g of a brown solid is recovered (yield=89%).

### Example 26

### Synthesis of 2-methyl-3-(glycinyl-aminoethyl)-1,4-naphthoquinone chlorohydrate

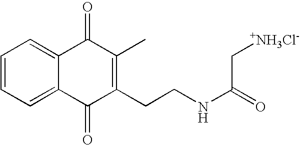

According to the operating protocol described for the compound of example 5, in a ml flask 0.030 g of the compound of example 16 (1 eq.; 0.077 mmol), is introduced, and 2 ml of CH₂Cl₂ and 2 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.021 g of an orange solid is recovered (yield=84%).

### Example 27

### Synthesis of 2-methyl-3-(N′-tert-butyloxycarbonyl-D-tryptophanyl-aminoethyl)-1,4-naphthoquinone

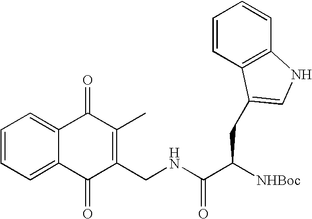

According to the coupling procedure described for the compound of example 7, 0.116 g of Boc-D-TrpOH (1.2 eq.; 0.38 mmol) 0.189 g of HBTU (1.5 eq.; 0.50 mmol), 0.054 g of HOBt (1.2 eq.; 0.40 mmol), 3 ml of CH₂Cl₂, and 0.30 ml of DIEA are used. 0.100 g of the compound of example 5 is then added and, after washing and purification of the product on a chromatography column with AcOEt:Cyclohexane (20:80) 0.109 g of an orange solid is obtained (yield=70%).

### Example 28

### Synthesis of 2-methyl-3-(N′-tert-butyloxycarbonyl-L-threoninyl(OBn)-aminoethyl)-1,4-naphthoquinone

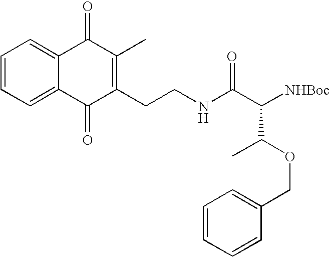

According to the coupling procedure described for the compound of example 7, 0.118 g of Boc-L-Thr(Bn)OH (1.27 eq.; 0.38 mmol) 0.180 g of HBTU (1.6 eq.; 0.47 mmol), 0.052 g of HOBt (1.3 eq.; 0.38 mmol), 3 ml of CH₂Cl₂ and 0.28 ml of DIEA are used. The activation is left to act for two hours under nitrogen. 0.100 g of the compound of example 6 is then added and, after washing and purification of the product on a chromatography column with AcOEt:Cyclohexane (20:80), 0.070 g of an orange solid is obtained (yield=45%).

### Example 29

### Synthesis of 2-methyl-3-(N′-tert-butyloxycarbonyl-D-tyrosinyl(OBn)-aminoethyl)-1,4-naphthoquinone

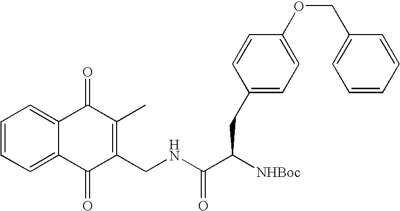

According to the coupling procedure described for the compound of example 7, 0.141 g of Boc-D-Tyr(Bn)OH (1.20 eq.; 0.38 mmol), 0.189 g of HBTU (1.57 eq.; 0.50 mmol), 0.054 g of HOBt (1.26 eq.; 0.40 mmol), 3 ml of CH₂Cl₂ and 0.30 ml of DIEA are used. The activation is left to act for two hours under nitrogen. 0.100 g of the compound of example 6 is then added and, after washing and purification of the product on a chromatography column with AcOEt:Cyclohexane (20:80), 0.065 g of an orange solid is obtained (yield=37%).

### Example 30

### Synthesis of 2-methyl-3-(D-tyrosinyl(OBn)-aminomethyl)-1,4-naphthoquinone chlorohydrate

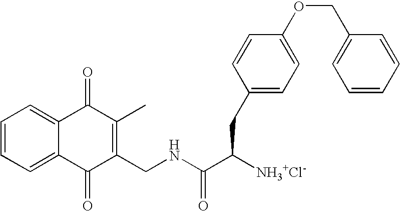

According to the operating protocol described previously for the compound of example 5, in a 10 ml flask 0.060 g of the compound of example 29 (1 eq.; 0.110 mmol) is introduced, and 1 ml of CH₂Cl₂ and 1 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.050 g of an orangey solid is recovered (yield=92%).

### Example 31

### Synthesis of 2-methyl-3-(D-tryptophanyl-aminomethyl)-1,4-naphthoquinone chlorohydrate

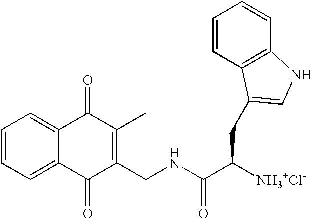

According to the operating protocol described previously for the compound of example 5, in a ml flask 0.104 g of the compound of example 27 (1 eq.; 0.213 mmol) is introduced, and 1 ml of CH₂Cl₂ and 1 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.084 g of an orangey solid is recovered (yield=93%).

### Example 32

### Synthesis of 2-methyl-3-(threoninyl-aminoethyl)-1,4-naphthoquinone chlorohydrate

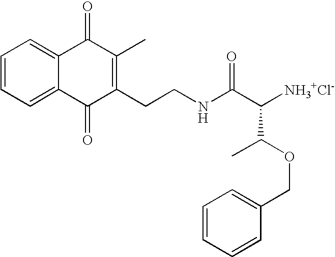

According to the operating protocol described previously for the compound of example 5, in a 10 ml flask 0.065 g of the compound of example 28 (1 eq.; 0.128 mmol) is introduced, and 0.5 ml of CH₂Cl₂ and 0.5 ml of TFA are added (in an ice bath). During evaporation the medium is taken up in toluene several times, and then the brown solid obtained is taken up in a solution of 1M HCl in Et₂O for 30 minutes. The solvent is evaporated and the operation is repeated with the hydrochloric acid solution in ether twice. After evaporation and drying, 0.051 g of an orangey solid is recovered (yield=90%).

### Example 33

### Synthesis of O-Trichloroethoxycarbonyl-plumbagone

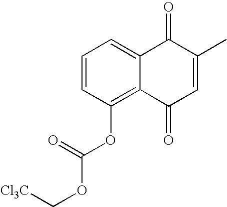

0.25 ml of pyridine (3 eq., 3.100 mmol) is added dropwise to a solution of 0.200 g of plumbagone (1 eq.; 1.060 mmol) in 5 ml of CH₂Cl₂ at 0° C. under argon. The initially orange mixture is stirred for 10 minutes and becomes brown. 0.18 ml of 2,2,2-trichloroethyl chloroformiate (1.25 eg., 1.300 mmol) is added and the dark orange mixture rapidly becomes a cloudy yellow. After 2 hours, the mixture is diluted in H₂O and extracted with AcOEt, dried on MgSO₄, filtered on Celite and concentrated at reduced pressure. 0.338 g of product is obtained in the form of pale yellow crystals (yield=87%).

### Example 34

### Synthesis of 3-(N′-tert-butyloxycarbonyl-2-aminoethyl)-O-trichloroethoxycarbonyl-plubagone

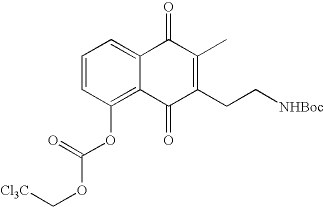

In a 10 ml flask, containing 1.100 g of the compound of example 33 (1 eq.; 0.28 mmol), 0.156 g of Boc-βAla (Aldrich Co) (3 eq.; 0.84 mmol) and 0.014 g of AgNO₃ (0.3 eq.l; 0.082 mmol) are introduced. The CH₃CN:water (2:1) mixture, that is to say 2.0 ml of CH₃Cn and 1.0 ml of H₂O, is added and the temperature of the mixture is raised to 65° C. The addition of 0.082 g of (NH₄)₂S₂O₈ (1.3 eq.; 0.360 mmol) dissolved in 1.5 ml of a CH₃CN:H₂O mixture takes place over 2 hours. The reaction medium is then kept stirred for one hour at 65° C., and then extraction is carried out with CH₂Cl₂ three times and the organic phases are washed just once with H₂O. The organic phases are dried on MgSO₄ and then evaporated. The product is purified on a chromatography column with AcOEt:Cyclohexane (10:90) as an eluant and 0.044 g of an orange oil is recovered (yield=31%).

### Example 35

### Synthesis of 3-(N′-tert-butyloxycarbonyl-2-aminoethyl)-plumbagone

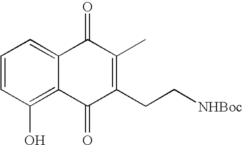

In a 10 ml flask, containing 0.200 g of plumbagone (Aldrich Co) (1 eq.; 1.06 mmol), 0.603 g of Boc-βAla (Aldrich Co) (3 eq.; 3.19 mmol) and 0.054 g of AgNO₃ (0.3 eq.; 0.32 mmol) are introduced. The CH₃CN:water (2:1) mixture is added, that is to say 2.0 ml of CH₃CN and 1.0 ml of H₂O, and the temperature of the mixture is raised to 65° C. The addition of 0.315 g of (NH₄)₂S₂O₈ (1.3 eq.; 1.38 mmol) dissolved in 1.5 ml of a CH₃CN:H₂O mixture takes place over 2 hours. The reaction medium is then kept stirred for one hour at 65° C. and then extraction is carried out with CH₂Cl₂ three times and the organic phases are washed just once with H₂O and 0.054 g of AgNO₃ (0.3 eq; 0.032 mmol). The organic phases are dried on MgSO₄ and then evaporated. The product is purified by chromatography column with AcOEt:Cyclohexane (0:100 to 20:80) as an eluant and 0.153 g of an orange oil is recovered (yield=43%).

### Example 36

### Synthesis of 3-(N′-tert-butyloxycarbonyl-2-aminoethyl)-O-acetyl-plumbagone

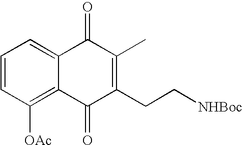

In a 10 ml flask. 0.183 g of the compound of example 35 (1 eq.; 0.55 mmol) is dissolved in 5.5 ml of CH₂Cl₂ under argon. 0.3 ml of Et₃N (3.9 eq; 2.16 mmol) is added and the mixture is stirred for 10 minutes at room temperature. 0.2 ml of acetyl chloride (5 eq.; 2.80 mmol) is added slowly at 0° C. and the mixture is stirred for 3 hours at room temperature. The reaction medium is washed with brine and then extracted with CH₂Cl₂. The organic phases collected together are dried on MgSO₄, filtered on Celite and concentrated at low pressure. 0.194 g of the product is obtained in the form of a brown solid (yield=94%).

### Example 37

### Synthesis of 3-aminoethyl-O-acetyl-plumbagone chlorhydrate

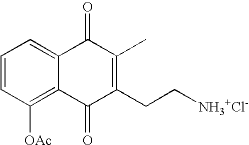

According to the operating protocol described previously for the compound of example 5, in a 10 ml flask, 0.190 g of the compound of example 36 is added (1 eq.; 0.509 mmol) is introduced, and 2 ml of CH₂Cl₂ and 2 ml of TFA are added (in an ice bath). During evaporation the medium is taken up several times in toluene, and then the brown solid obtained is taken up in a solution of 1M HCl in Et₂O for 30 minutes. The solvent is evaporated and the operation is repeated with the solution of hydrochloric acid in ether twice. After evaporation and drying, 0.113 g of a brown solid is recovered (yield=83%).

After suitable synthetic transformations similar to those described for the series of compounds of examples 1 to 26 above, and which will become apparent to a person skilled in the art from a reading of the present document, this intermediate can give rise to a compound of formula I, where Z is linked to the X radical via an amide bond, by coupling with a suitable amino acid. The compound obtained would for example comply with the following formula if the aforementioned intermediate is coupled with an α-amino acid:

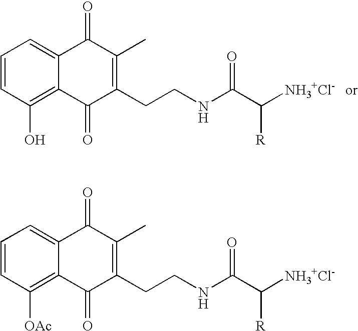

(R designating the lateral chain of the amino acid)

### Example 38

### Synthesis of 2-(N′-tert-butyloxycarbonyl-aminoethyl)-5-hydroxy-1,4-naphthoquinone and 3-(N′-tert-butyloxycarbonyl-aminoethyl)-5-hydroxy-1,4-naphthoquinone

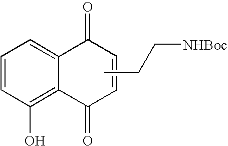

In a 25 ml flask containing 0.348 g of juglone (Aldrich Co) (1 eq.; 2.00 mmol), 1.135 g of Boc-βAla (Aldrich Co) (3 eq.; 6.00 mmol) and 0.102 g of AgNO₃ (0.3 eq.; 0.66 mmol) are introduced. The Ch₃CN:water (2:1) mixture, that is to say 5.0 ml of CH₃CN and 2.5 ml of H₂O, is added and the temperature of the mixture is raised to 65° C. The addition of 0.593 g of (NH₄)₂S₂O₈ (1.3 eq.; 2.60 mmol) dissolved in 3.0 ml of a CH₃CN:H₂O mixture takes place over two hours. The reaction medium is then kept under stirring for one hour at 65° C., and then extraction is carried out with CH₂Cl₂ three times and the organic phases are washed with H₂O just once. The organic phases are dried on MgSO₄ and then evaporated. The product is purified by chromatography column with a CH₂Cl₂ eluant and 0.123 g of a mixture of two regioisomers are recovered in a ratio of 3:1 in the form of an orange oil (yield=19%). After suitable synthetic transformations, similar to those described for the series of compounds in examples 1 to 26 above, in which it will become obvious to a person skilled in the art from a reading of the present document, this intermediate can give rise to compound of formula I, where Z is linked to the X radical via an amide bond, by coupling with a suitable amino acid. The compound obtained would for example comply with the following formula if the aforementioned intermediate is coupled with an α-amino acid:

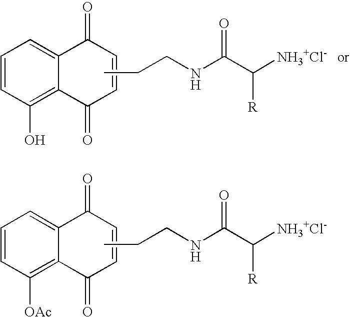

(R designating the lateral chain of the amino acid).

### Example 39

### Synthesis of 2,3-bis-(N′-tert-butyloxycarbonyl-aminoethyl)-5-hydroxy-1,4-naphthoquinone

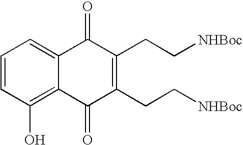

In a 5 ml flask containing 0.100 g of the mixture of compounds of example 38 (1 eq.; 0.315 mmol), 0.179 g of Boc-βAla (Aldrich Co) (3 eq.; 0.95 mmol) and 0.016 g AgNO₃ (0.3 eq.; 0.095 mmol) are introduced. The CH₃CN:water (2:1) mixture, that is to say 1.0 ml of CH₃CN and 0.5 ml of H₂O, is added and the temperature of the mixture is raised to 65° C. The addition of 0.094 g of (NH₄)₂S₂O₈ (1.3 eq.; 0.41 mmol) dissolved in 1.0 ml of a CH₃CN:H₂O mixture, takes place over two hours. The reaction medium is then kept under stirring for one hour at 65° C., and then extraction is carried out with CH₂Cl₂ three times and the organic phases are washed with H₂O just once. The organic phases are dried on MgSO₄ and then evaporated. The product is purified by chromatography column with a CH₂Cl₂ eluant and 0.067 g of an orange oil (yield=46%) is recovered.

### Example 40

### Synthesis of 2-methyl-)N′-acetyl-1′-methyl-aminomethyl)-1,4-naphthoquinone

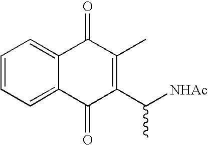

In a 10 ml flask containing 0.200 g of menadione (1 eq.: 1.16 mmol), 0.456 g of N—Ac-D,L-Ala (Aldrich Co) (3 eq.; 3.48 mmol) and 0.059 g of AgNO₃ (0.3 eq.; 0.035 mmol) is introduced. The CH₃CN:water (2:1) mixture, that is to say 3.0 ml of CH₃CN and 1.5 ml of H₂O, is added and the temperature of the mixture is raised to 65° C. The addition of 0.344 g of (NH₄)₂S₂O₈ (1.3 eq.; 0.51 mmol) dissolved in 1.0 ml of a CH₃CN:H₂O mixture takes place over two hours. The reaction medium is then kept under stirring for one hour at 65° C., and then extraction is carried out with CH₂Cl₂ three times and the organic phases are washed with H₂O just once. The organic phases are dried on MgSO₄ and then evaporated. The product is purified on a chromatography column with CH₃OH:CH₂Cl₂ as an eluant (from 0:100 to 5:95) and 0.048 g of an orange oil is recovered (yield=17%).

After suitable synthetic transformations, similar to those described for the series of compounds in examples 1 to 26 above, and which will become obvious to a person skilled in the art from a reading of the present document, this intermediate can give rise to compound of formula I, where Z is linked to the X radical via an amide bond, by coupling with a suitable amino acid. The compound obtained would for example comply with the following formula if the aforementioned intermediate is coupled with an α-amino acid:

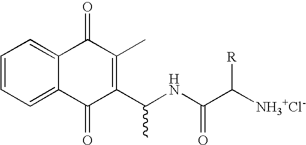

(R designating the lateral chain of the amino acid).

### Example 41

### Synthesis of 2-methyl-)N′-acetyl-1′-benzyl-aminomethyl)-1,4-naphthoquinone

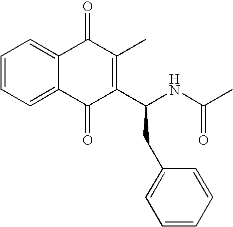

In a 10 ml flask containing 0.200 g of menadione (1 eq.: 1.16 mmol), 0.722 g of N—Ac-L-Phe (Aldrich Co) (3 eq.; 3.48 mmol) and 0.065 g of AgNO₃ (0.3 eq.; 0.038 mmol) is introduced. The CH₃CN:water (2:1) mixture, that is to say 3.0 ml of CH₃CN and 1.5 ml of H₂O, is added and the temperature of the mixture is raised to 65° C. The addition of 0.375 g of (NH₄)₂S₂O₈ (1.3 eq.; 1.64 mmol) dissolved in 3.0 ml of a CH₃CN:H₂O mixture, takes place over two hours. The reaction medium is then kept under stirring for one hour at 65° C., and then extraction is carried out with CH₂Cl₂ three times and the organic phases are washed with H₂O just once. The organic phases are dried on MgSO₄ and then evaporated. The product is purified on a chromatography column with CH₃OH:CH₂Cl₂ as an eluant (from 0:100 to 5:95) and 0.387 g of an orange oil is recovered (yield=22%).

After suitable synthetic transformations, similar to those described for the series of compounds in examples 1 to 26 above, and which will become obvious to a person skilled in the art from a reading of the present document, this intermediate can give rise to compound of formula I, where Z is linked to the X radical via an amide bond, by coupling with a suitable amino acid. The compound obtained would for example comply with the following formula if the aforementioned intermediate is coupled with an α-amino acid:

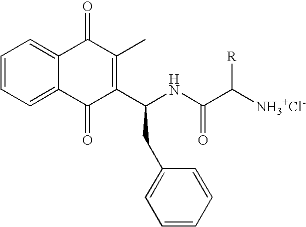

(R designating the lateral chain of the amino acid).

### Example 42

### Synthesis of 2-methyl-1-4-naphthoquinone methyl 3-propanoate

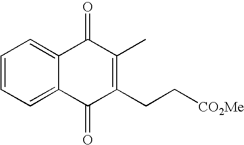

In a 100 ml flask containing 1.000 g of menadione (Aldrich Co) (1 eq.; 5.81 mmol), 2.300 of methyl monoester of succinic acid (Aldrich Co) (3 eq.; 17.40 mmol) and 0.300 g of AgNO₃ (0.3 eq.; 1.80 mmol) are introduced. The CH₃CN:water (2:1) mixture is added, that is to say 30.0 ml of CH₃CN and 15.0 ml of H₂O, and the temperature of the mixture is raised to 65° C. The addition of 1.730 g of (NH₄)₂S₂O₈ (1.3 eq.; 7.60 mmol) dissolved in 30.0 ml of a CH₃CN:H₂O mixture takes place over 2 hours. The reaction medium is then kept stirred for one hour at 65° C., and then extraction is carried out with CH₂Cl₂ three times and the organic phases are washed just once with H₂O. The organic phases are dried on MgSO₄ and then evaporated. The product is purified on a chromatography column with CH₃OH; CH₂Cl₂ eluant (gradient from 0:100 to 5:95) and 0.875 g of an orange oil is recovered (yield=58%).

### Example 43

### Synthesis of methyl 3-propanoate 1,4-naphthoquinone

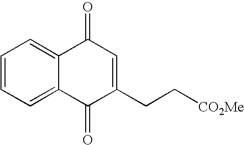

In a 10 ml flask containing 0.097 g of 1,4-naphthonquinone (Acros Co) (1 eq.; 0.61 mmol), 0.242 of methyl monoester of succinic acid (Aldrich Co) (3 eq.; 1.83 mmol) and 0.031 g of AgNO₃ (0.3 eq.; 0.18 mmol) are introduced. The CH₃CN:water (2:1) mixture is added, that is to say 3.0 ml of CH₃CN and 1.5 ml of H₂O, and the temperature of the mixture is raised to 65° C. The addition of 0.181 g of (NH₄)₂S₂O₈ (1.3 eq.; 0.79 mmol) dissolved in 3.0 ml of a CH₃CN:H₂O mixture takes place over 2 hours. The reaction medium is then kept stirred for one hour at 65° C. and then extraction is carried out with CH₂Cl₂ three times and the organic phases are washed just once with H₂O. The organic phases are dried on MgSO₄ and then evaporated. The produce is purified on a chromatography column with CH₃OH; CH₂Cl₂ eluant (gradient from 0:100 to 5:95) and 0.042 g of an orange oil is recovered (yield=28%).

### Example 44

### Synthesis of 2-methyl-3-propanoyl-1,4-naphthoquinone

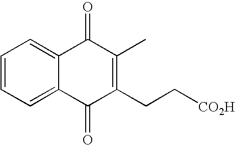

In a 5 ml flask, 0.120 g of the compound of example 42 (1 eq.; 0.46 mmol) is weighed. 0.5 ml of an aqueous solution of 1M HCl (1.1 eq.; 0.5 mmol) is added. The reaction medium is stirred at room temperature for one night. After addition of CH₂Cl₂ and extraction of the aqueous phase by CH₂Cl₂, the organic phases collected together are dried on MgSO₄, filtered and concentrated at reduced pressure. The product is purified by column chromatography with CH₃OH:CH₂Cl₂ (gradient from 0:100 to 10:90) as an eluant and 0.053 g of a yellow oil is recovered (yield=46%).

### Example 45

### Synthesis of 3-propanoyl-1,4-naphthoquinone

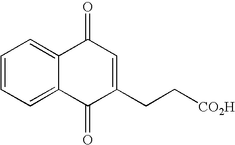

In a 5 ml flask, 0.120 g of the compound of example 43 (1 eq.; 0.49 mmol) is weighed. 0.55 ml of an aqueous solution of 1M HCl (1.1 eq.; 0.55 mmol) is added. The reaction medium is stirred at room temperature for one night. After addition of CH₂Cl₂ and extraction of the aqueous phase by CH₂Cl₂, the organic phases collected together are dried on MgSO₄, filtered and concentrated at reduced pressure. The product is purified by column chromatography with CH₃OH:CH₂Cl₂ (gradient from 0:100 to 10:90) as an eluant and 0.048 g of a yellow oil is recovered (yield=42%).

After suitable synthetic transformations that will become obvious to a person skilled in the art from a reading of the present document, the intermediates in examples 44 and 45 may give rise to compounds of formula I where Z is linked to the X radical via a retro-inverso amide bond, by coupling with a suitable amino acid. The compounds obtained will for example comply with the following formulae if the aforementioned intermediates were coupled with an α-amino acid:

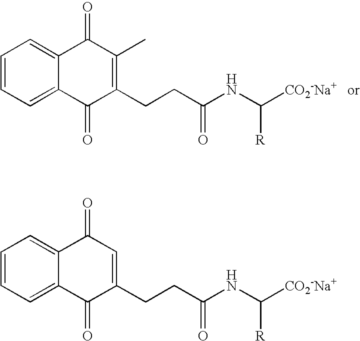

(R designating the lateral chain of the amino acid).

### Example 46

### Synthesis of 2-methyl-3-propanol-1,4-naphthoquinone

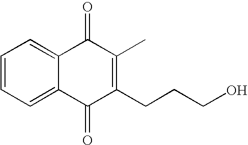

In a 10 ml flask, 0.100 g of the compound of example 42 (1 eq.; 0.39 mmol) is weighed and dissolved in 5 ml of ethanol. 0.100 g of sodium borohydride (6.7 eq.; 2.6 mmol) is added at 0° C. and the mixture is stirred to reflux for seven hours. The reaction medium is cooled at room temperature before being poured into 10 ml of iced water and then acidified with an aqueous solution of 1% sulphuric acid and extracted with CHCl₃. The organic phases collected together are dried on Na₂SO₄, filtered and concentrated at reduced pressure. 0.043 g of product is obtained in the form of a pale yellow oil (yield=48%).

### Example 47

### Synthesis of 3-propanol-1,4-naphthoquinone

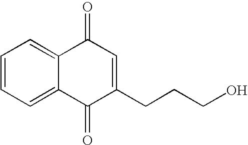

In a 10 ml flask, 0.100 g of the compound of example 43 (1 eq.; 0.41 mmol) is weighed and dissolved in 5 ml of ethanol. 0.100 g of sodium borohydride (6.7 eq.; 2.6 mmol) is added at 0° C. and the mixture is stirred to reflux for seven hours. The reaction medium is cooled at room temperature before being poured into 10 ml of iced water and then acidified with an aqueous solution of 1% sulphuric acid and extracted with CHCl₃. The organic phases collected together are dried on Na₂SO₄, filtered and concentrated at reduced pressure. 0.040 g of product is obtained in the form of a pale yellow oil (yield=45%).

After suitable synthetic transformations that will become obvious to a person skilled in the art from a reading of the present document, the intermediates in examples 46 and 47 may give rise to compounds of formula I where Z is linked to the X radical via an ester bond, by coupling with a suitable amino acid. The compounds obtained will for example comply with the following formula if the aforementioned intermediaries were coupled with an α-amino acid:

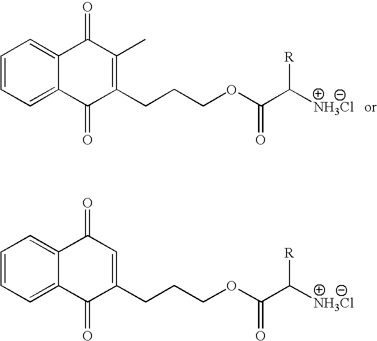

(R designating the lateral chain of the amino acid).

### Example 48

### Synthesis of 3-(N′-tert-butyloxycarbonyl-2-aminomethyl)-1,4-naphthoquinone

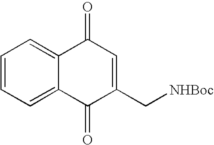

In a 100 ml flask, 0.950 g of 1,4-naphthoquinone (1 eq., 6.0 mmol), 3.150 g (3 eq., 18.0 mmol) of Boc-Gly (Aldrich Co), and 0.306 g of AgNO₃ (0.3 eq.; 1.8 mmol) are weighed. The acetonitrile:water mixture (ratio 7:3), that is to say 30.0 ml of CH₃CN and 15.0 ml of H₂O is added and the temperature of the mixture is raised to 65° C. The addition of 1.78 g of (NH₄)S₂O₈ (1.3 eq.; 7.8 mmol) dissolved in 15 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by column chromatography with CH₃OH:CH₂Cl₂ (from 0:100 to 5:95) as an eluant and 0.812 g of an orange oil is obtained (yield=47%).

### Example 49

### Synthesis of 3-(N′-tert-butyloxycarbonyl-2-aminoethyl)-1,4-naphthoquinone

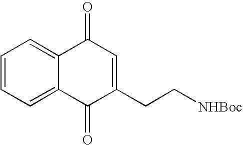

In a 100 ml flask, 0.950 g of 1,4-naphthoquinone (1 eq., 6.0 mmol), 3.410 g (3 eq., 18.0 mmol) of Boc-βAla (Aldrich Co), and 0.306 g of AgNO₃ (0.3 eq.; 1.8 mmol) are weighed. The acetonitrile:water mixture (ratio 7:3), that is to say 30.0 ml of CH₃CN and 15.0 ml of H₂O is added and the temperature of the mixture is raised to 65° C. The addition of 1.78 g of (NH₄)S₂O₈ (1.3 eq.; 7.8 mmol) dissolved in 15 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by column chromatography with CH₃OH:CH₂Cl₂ (from 0:100 to 5:95) as an eluant and 0.488 g of an orange oil is obtained (yield=27%).

### Example 50

### Synthesis of 2,3-bis-(N′-tert-butyloxycarbonyl-2-aminomethyl)-1,4-napthoquinone

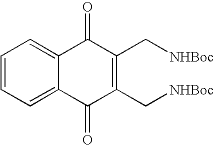

### Method A:

In a 100 ml flask, 0.475 g of 1,4-naphthoquinone (1 eq., 3.0 mmol), 1,580 g (3 eq., 9.0 mmol) of Boc-Gly (Aldrich Co), and 0.153 g of AgNO₃ (0.3 eq.; 0.9 mmol) are weighed. The acetonitrile:water mixture (ratio 7:3), that is to say 20.0 ml of CH₃CN and 10.0 ml of H₂O, is added and the temperature of the mixture is raised to 65° C. The addition of 0.890 g of (NH₄)S₂O₈ (1.3 eq.; 3.9 mmol) dissolved in 15 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt Cyclohexane (from 0:100 to 30:70) as an eluant and 0.240 g of an orange oil is obtained (yield=19%).

### Method B;

In a 25 ml flask, 0.300 g of the compound of example 48 (1 eq.; 1.04 mmol), 0.549 g (3 eq.; 3.13 mmol) of Boc-Gly (Aldrich Co) and 0.053 g of AgNO₃ (0.3 eq.; 0.31 mmol) are weighed. The acetonitrile:water mixture (ratio 7:3), that is to say 7.0 ml of CH₃CN and 3.0 ml of H₂O, is added and the temperature of the mixture is raised to 65° C. The addition of 0.310 g of (NH₄)S₂O₈ (1.3 eq.; 1.36 mmol) dissolved in 5 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 0:100 to 30:70) as an eluant and 0.292 g of an orange oil is obtained (yield=67%).

### Example 51

### Synthesis of 2-(N′-tert-butyloxycarbonyl-2-aminomethyl)-3-(N′-tert-butyloxycarbonyl-2-aminoethyl)-1,4 naphthoquinone

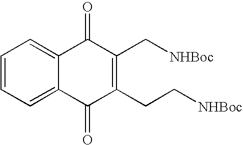

In a 25 ml flask, 0.287 g of the compound of example 48 (1 eq.; 1.0 mmol), 0.568 g (3 eq.; 3.0 mmol) of Boc-βAla (Aldrich Co) and 0.051 g of AgNO₃ (0.3 eq.; 0.30 mmol) are weighed. The acetonitrile:water mixture (ratio 7:3), that is to say 5.0 ml of CH₃CN and 2.5 ml of H₂O, is added and the temperature of the mixture is raised to 65° C. The addition of 0.297 g of (NH₄)S₂O₈ (1.3 eq.; 1.30 mmol) dissolved in 4.5 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 10:90 to 70:30) as an eluant and 0.330 g of an orange oil is obtained (yield=76%).

### Example 52

### Synthesis of 2,3-bis-(N′-tert-butyloxycarbonyl-2-aminoethyl)-1,4-naphthoquinone

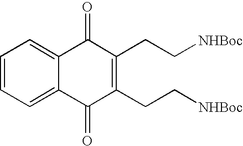

### Method A:

In a 100 ml flask, 0.475 g of 1,4-naphthoquinone (1 eq., 3.0 mmol), 1,703 g (3 eq., 9.0 mmol) of Boc-βAla (Aldrich Co), and 0.153 g of AgNO₃ (0.3 eq.; 0.9 mmol) are weighed. The acetonitrile:water mixture (ratio 7:3), that is to say 20.0 ml of CH₃CN and 10.0 ml of H₂O, is added and the temperature of the mixture is raised to 65° C. The addition of 0.890 g of (NH₄)S₂O₈ (1.3 eq.; 3.9 mmol) dissolved in 15 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 0:100 to 30:70) as an eluant and 0.160 g of an orange oil is obtained (yield=12%).

### Method B:

In a 25 ml flask, 0.300 g of the compound of example 49 (1 eq.; 0.99 mmol), 0.565 g (3 eq.; 2.99 mmol) of Boc-βAla (Aldrich Co) and 0.051 g of AgNO₃ (0.3 eq.; 0.30 mmol) are weighed. The acetonitrile:water mixture (ratio 7:3), that is to say 7.0 ml of CH₃CN and 3.0 ml of H₂O is added and the temperature of the mixture is raised to 65° C. The addition of 0.295 g of (NH₄)S₂O₈ (1.3 eq.; 1.29 mmol) dissolved in 5 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 0:100 to 30:70) as an eluant and 0.208 g of an orange oil is obtained (yield=47%).

### Example 53

### Synthesis of 2-(N′-tert-butyloxycarbonyl-3-aminopropyl)-1,4-naphthoquinone

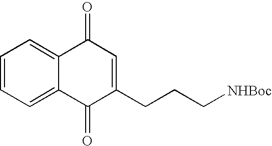

In a 50 ml flask, 0.545 g of 1,4-naphthoquinone (1 eq., 3.45 mmol), 2.10 g of N-Boc-4-aminobutyric acid (3 eq., 10.3 mmol), and 0.176 g of AgNO₃ (0.3 eq.; 1.04 mmol) are weighed. The acetonitrile:water mixture (ratio 7:3), that is to say 16.0 ml of CH₃CN and 8.0 ml of H₂O, is added and the temperature of the mixture is raised to 65° C. The addition of 1.020 g of (NH₄)S₂O₈ (1.3 eq.; 4.47 mmol) dissolved in 12.0 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 10:90 to 70:30) as an eluant and 0.142 g of an orange oil is obtained (yield=13%).

### Example 54

### Synthesis of 2-(N′-tert-butyloxycarbonyl-2-aminomethyl)-3-(N′-tert-butyloxycarbonyl-3-aminopropyl)1,4-naphthoquinone

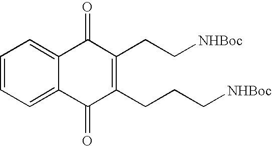

In a 10 ml flask containing 0.071 g of the compound of example 53 (1 eq., 0.23 mmol), 0.128 g (3 eq., 0.68 mmol) of Boc-βAla (Aldrich Co), and 0.012 g of AgNO₃ (0.3 eq.; 0.071 mmol) are weighed. The acetonitrile:water mixture (ratio 7:3), that is to say 2.0 ml of CH₃CN and 1.0 ml of H₂O, is added and the temperature of the mixture is raised to 65° C. The addition of 0.067 g of (NH₄)S₂O₈ (1.3 eq.; 0.29 mmol) dissolved in 1.5 ml of a CH₃CN:H₂O mixture takes place over two hours. The reaction medium is then kept under stirring for one hour at 65° C., then extraction is carried out with CH₂Cl₂ three times and the etherated phases are washed with H₂O just once. The organic phases are dried on MgSO₄ and evaporated. The product is purified by chromatography on a column with AcOEt:Cyclohexane (from 10:90 to 70:30) as an eluant and 0.045 g of an orange oil is obtained (yield=43%).

### Example 55

### Synthesis of 2-aminomethyl-1,4-naphthoquinone chlorhydrate

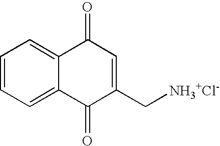

According to the operating protocol described previously for the compound of example 5, in a 10 ml flask, 0.100 g of the compound of example 49 is added (1 eq.; 0.348 mmol) is introduced, and 1 ml of CH₂Cl₂ and 1 ml of TFA are added (in an ice bath). During evaporation the medium is taken up several times in toluene, and then the brown solid obtained is taken up in a solution of 1M HCl in Et₂O for 30 minutes. The solvent is evaporated and the operation is repeated with the solution of hydrochloric acid in ether twice. After evaporation and drying, 0.074 g of an orangey solid is recovered (yield=94%).

### Example 56

### Synthesis of 2-aminoethyl-1,4-naphthoquinone chlorhydrate

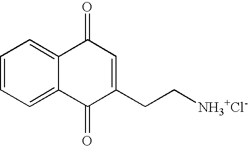

According to the operating protocol described previously for the compound of example 5, in a 10 ml flask, 0.100 g of the compound of example 49 is added (1 eq.; 0.332 mmol) is introduced, and 1 ml of CH₂Cl₂ and 1 ml of TFA are added (in an ice bath). During evaporation the medium is taken up several times in toluene, and then the brown solid obtained is taken up in a solution of 1M HCl in Et₂O for 30 minutes. The solvent is evaporated and the operation is repeated with the solution of hydrochloric acid in ether twice. After evaporation and drying, 0.073 g of an orangey solid is recovered (yield=92%).

### Example 57

### Synthesis of Sulfonamides

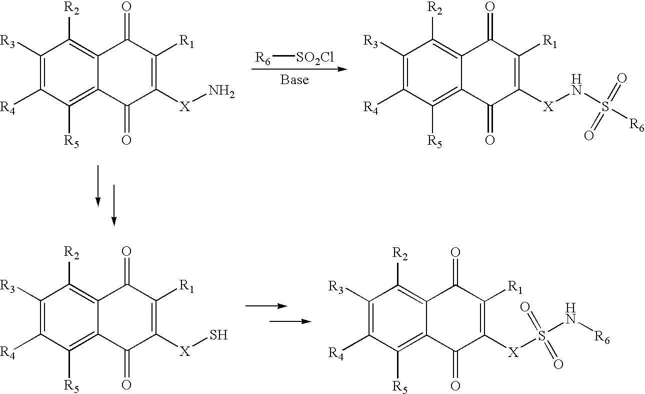

The sulphonamide bond is interesting since it is isosteric of the amide.

As illustrated above, the formation of the sulphonamide bond can be achieved using a naphthoquinone functionalised in the form of a terminal amine (or in the form of an ammonium salt) like the compounds of examples 5-6 or 55-56, which can react with a sulfonyl chloride in order to lead to a sulphonamide.

It is also possible to envisage an inverse bond in which the naphthoquinone would be functionalised by in examples 46-47) or an amine function. This thiol can be oxidised in order to lead to a sulfonic acid, which can then be activated in the form of a sulfanide chloride before reacting with a suitable functionalised amine.

### Example 58

### Synthesis of retro-inverso amide bonds, and esters

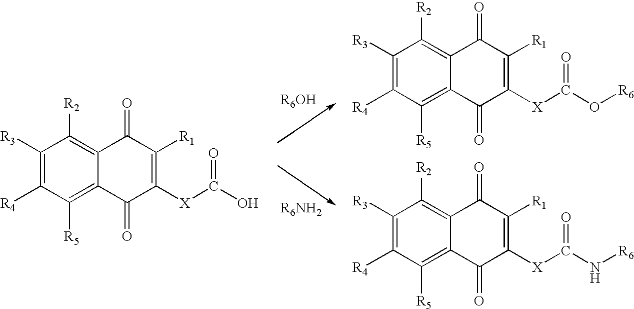

Among the isosteres of the amide bond, the retro-inverso bond contains the same functional groups and therefore remains very similar to the conventional amide.

The naphthoquinone compound is functionalised in the form of a carboxylic acid (as in examples 44 or 45), which can be activated according to the procedure described for example 7 and then put together with a suitably functionalised amine, or an amino acid protected on its carboxylic function in order to lead to a retro-inverso amide. For example, if this amino acid is an a alanine, in the above diagram R₆ is CH—CO₂GP, where GP is a protective group of the carboxylic acid function, the amide bond is reversed with respect to the compound of example 7.

From the compounds of examples 44 or 45, it is possible, according to the same procedure described above, to produce esters, by making the activated carboxylic acid react with a compound comprising an alcohol function. R₆OH can be the lateral chain of an amino acid such as Ser or Thr, the amine and carboxylic acid functions of which would be protected, or R₆ could be of the CH—NHGP type, and would therefore be a previously reduced amino acid derivative.

It is also possible to produce carbonates from compounds like those of examples 46 or 47, using an R₆OH alcohol as described previously, R₆ could be an amino acid derivative (or other thing), according to the formula Naphthoquinone-spacer-O—(CO)—O—R₆.

It is possible to imagine producing thioester and thioamide bonds according to the methods described above.

### Example 59

### Synthesis of Thioamides and Thioesters

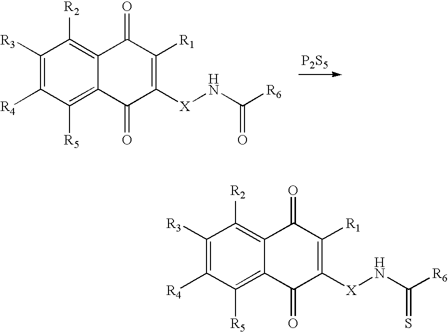

As illustrated above, the formation of the thioamide bond can be achieved by the transformation of an amide bond (or retro-inverso amide) in the presence of sulphur pentasulfide. It is also possible to achieve this same type of bond according to other methods, for example by generating a thioaldehyde in the presence of a suitably substituted amine (an amino acid protected on its carboxylic function, and on its lateral chain for example).

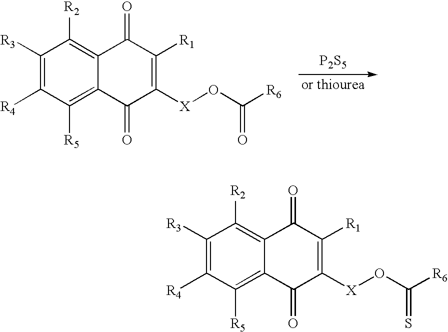

It is possible to envisage the transformation of an ester bond according to the same method or in the presence of thiourea. But in this case also, numerous different procedures exist, such as the reaction between a suitability substituted alcohol (which may be an amino acid derivative for example) and a thioamide in which the amine group (a nitrated aromatic for example) takes the place of a starting group that will be displaced by the aforementioned alcohol.

Various chemical transformations allowing the preparation of compounds illustrated in the above examples will become clear to a person skilled in the art from a reading of the text of the application, and in the light of all the knowledge available to him in the field of organic chemistry in general, and more particularly synthetic transformations, as listed in numerous reference works, such as for example:
1. “Advanced Organic Chemistry—Reactions, Mechanisms and Structure”, Jerry March, John Wiley & Sons, 5^{th} edition, 2001;
2. “Comprehensive Organic Transformations, A Guide to Functional Group Preparations”, Richard C Larock, VCH Publishers, 2^{nd} edition, 1999;
3. “Benzotriazole-Assisted Thioacylation” Katritzky, A R; Witek, R M; Rodriguez-Garcia, V; Mohapatra, P P; Rogers, J W; Cusido, J; Abdel-Fattah, A A A; Steel, P J., J Org. Chem., 2005 20(70): 7866-7881.

### Example 60

### Biological Tests

The biological tests on activity (Sub-G1) relate to measurement of apoptosis. For this purpose, a fluorescent tracer, a DNA intercalator, makes it possible to display the fragmentation of the DNA. This fragmentation of the DNA results from the induction of the apoptotic phenomenon in the cells. The cells are incubated in the presence of various concentrations of the compounds presented above. The measurement of the apoptosis in the cells is monitored by measuring the fluorescence, thus making it possible to determine the activity of the compounds in micromoles (μm). The activity tests (sub-G1) on certain compounds are presented below.

| | Compound | Activity (sub-Gl) μM |
|---|---|---|
| 1 | Compound of example 17 | 13.3 |
| 2 | Compound of example 18 | 11.8 |
| 3 | Compound of example 19 | 12 |
| 4 | Compound of example 20 | 12.2 |

The biological tests show that the compounds complying with formula (I) (entries 1, 2, 3, 4) have a pro-apoptotic activity.

### Examples 61

### Biological Tests

The activities are measured by multiplexed flow cytometry tests, by coupling the measurement of apoptosis and cell proliferation. The cell proliferation is measured by monitoring the dilution of a specific fluorescent tracer during the cell divisions:
the cells are incubated in the presence of several concentrations of compounds, then
the measurements of apoptosis and cell proliferation are made simultaneously by the flow cytometry technique; specimen dose/response curves for the action of the compounds on apoptosis and on cell proliferation are obtained. It is therefore possible to demonstrate the action of the compounds on the phenomena of apoptosis and cell proliferation and to determine the activity of the compounds in micromoles (μm).

The measurements of the activities of certain compounds are presented below.

| | | Activity | Activity |
|---|---|---|---|
| | | (prolif at 24 h) | (sub-Gl) |
| | Compound | μM | μM |
| 5 | Compound of example 22 | 11.5 | 22.8 |
| 6 | Compound of example 23 | 11.1 | 12.1 |
| 7 | Compound of example 25 | 6.5 | 13 |
| 8 | Compound of example 26 | 12.3 | 11.8 |

These biological tests show clearly that the compounds complying with formula (I) (entries 5, 6, 7, 8) have a pro-apoptotic and anti-proliferative activity.

1. An isolated compound of the following formula (I), or one of its pharmaceutically acceptable salts, by way of medication: 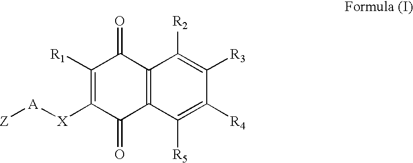
in which
A is —O—, —S—, —SO₂—, —C(═S)—, —CO— or a chemical function such that Z′ and X′ are linked by a bioisosteric bond of the amine function,
R1 represents a hydrogen atom; a halogen atom; a hydroxyl function, possibly substituted; a C₁₋₁₈alkyl, C₂₋₁₈alkene or C₂₋₁₈alkyne radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups; an C₆₋₁₀aryl C₁₋₆alkyl or C₁₋₆alkyl C₆₋₁₀aryl radical, possibly substituted, in particular by one or more amino, carboxylic acid, carboxylic acid derivative, C₁₋₁₈alkoxy, C₆₋₁₀aryl or hydroxy groups; a nitro function; or an —X′-A′-Z′ function in which;
(i) X′ represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom,
(ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, and
(iii) Z′ represents an amino acid residue, in particular D or L, natural or synthetic, in particular an α, β or γ amino acid residue, the terminal amine or carboxyl function (ie not linked to X′) and any lateral chemical functions of Z′ being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R2, R3, R4 and R5 represent independently of one another a hydrogen atom; a halogen atom; a hydroxyl function, possibly substituted, a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; or a nitro function;
or R2 and R3, R3 and R4 and/or R4 and R5 form together a ring or a heterocyclic compound, possibly substituted,
X represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom, and
Z represents an amino acid residue, in particular D or L, natural or synthetic, in particular an α, β or γ amino acid residue, the terminal amine or carboxyl function (ie not linked to X) and any lateral chemical functions of Z being protected or not, where Z is linked to the X radical via a retro-inverso amide bond, an ester bond, or a sulphonamide bond, a thioester bond or a thioamide bond, or a bioisosteric bond of the amide bond, resulting from the coupling of X with a terminal aldehyde or alcohol function of Z resulting from the reduction of the terminal carboxyl function of the Z amino acid residue;
or in which
A is —NY—; and
(a)
R1 represents a hydrogen atom; a halogen atom; a hydroxyl function, possibly substituted; a C₁₋₁₈alkyl, C₂₋₁₈alkene or C₂₋₁₈alkyne radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a nitro function; or an —X′-A′-Z′ function in which;
(i) X′ represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom,
(ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, and
(iii) Z′ represents an amino acid residue, in particular D or L, natural or synthetic, in particular an α, β or γ amino acid residue, the terminal amine or carboxyl function (ie not linked to X′) and any lateral chemical functions of Z′ being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R3, R4 and R5 represent independently of one another a hydrogen atom; a halogen atom; a hydroxyl function, possibly substituted, a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀aryl radical, possibly substituted, in particular by one or more amino, carboxylic acid, carboxylic acid derivative, C₁₋₁₈alkoxy, C₆₋₁₀ aryl or hydroxyl groups; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; or a nitro function;
R2 represents, independently of R1, R2, R3, R4 and R5, a hydrogen atom; a halogen atom; a substituted hydroxyl function; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxyl groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; or a nitro function;
or R2 and R3, R3 and R4 and/or R4 and R5 form together a ring or a heterocyclic compound, possibly substituted,
X represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom,
Y represents a hydrogen atom or a protective group, and
Z represents an amino acid residue, in particular D or L, natural or synthetic, in particular an α, β or γ amino acid residue, the terminal amine function (ie not linked to X) and any lateral chemical functions of Z being protected or not, where Z is linked to the X radical via an amide bond;
(b)
R1 represents a hydrogen atom; a halogen atom; a hydroxyl function, possibly substituted; a C₁₋₁₈alkyl, C₂₋₁₈alkene or C₂₋₁₈alkyne radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a nitro function; or an —X′-A′-Z′ function in which;
(i) X′ represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom,
(ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, and
(iii) Z′ represents an amino acid residue, in particular D or L, natural or synthetic, in particular an α, β or γ amino acid residue, the terminal amine or carboxyl function (ie not linked to X′) and any lateral chemical functions of Z being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R3, R4 and R5 represent independently of one another a hydrogen atom; a halogen atom; a hydroxyl function, possibly substituted, a C₆₋₁₀aryl, C₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; or a nitro function;
R2 represents a hydroxyl function;
or R3 and R4 and/or R4 and R5 form together a ring or a heterocyclic compound, possibly substituted,
X represents a divalent radical, in particular chosen from C₁₋₁₈alkyls, C₂₋₁₈alkenes or C₂₋₁₈alkynes, linear or cyclic, not substituted, possibly interrupted by a heteroatom,
Y represents a hydrogen atom or a protective group, and
Z represents an amino acid residue, in particular D or L, natural or synthetic, in particular an α, β or γ amino acid residue, the terminal amine function (ie not linked to X) and any lateral chemical functions of Z being protected or not, where Z is linked to the X radical via an amide bond;
or in which:
(c)
R1 represents a halogen atom; a hydroxyl function, possibly substituted, a C₁₋₁₈alkyl, C₂₋₁₈alkene, or C₂₋₁₈alkyne radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a nitro function; or an —X′-A′-Z′ function in which;
(i) X′ represents a divalent radical, in particular chosen from C₁₋₁₈alkyls, C₂₋₁₈alkenes or C₂₋₁₈alkynes, linear, branched or cyclic, substituted or not, chiral or non-chiral, possibly interrupted by a heteroatom,
(ii) A′ is —O—, —S—, —NY′—, —SO₂—, —C(═S)—, —CO— or a chemical function such that Z′ and X′ are linked by a bioisosteric bond of the amide function, where Y′ represents a hydrogen atom or a protective group, and
(iii) Z′ represents an amino acid residue, in particular D or L, natural or synthetic, in particular an α, β or γ amino acid residue, the terminal amine or carboxyl function (ie not linked to X′) and any lateral chemical functions of Z being protected or not, where Z′ is linked to the X′ radical via an amide bond, a retro-inverso amide bond, an ester bond, or a sulphonamide bond, a thioester bond or a thioamide bond, or a bioisosteric bond of the amide bond, resulting from the coupling of X′ with a terminal aldehyde or alcohol function of Z′ resulting from the reduction of the terminal carboxyl function of the Z′ amino acid residue;
R2 represents a hydroxyl function;
R3, R4 and R5 represent independently of one another a hydrogen atom; a halogen atom; a hydroxyl function, possibly substituted, a C₆₋₁₀aryl, C₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; a C₆₋₁₀aryl radical, possibly substituted, in particular by one or more amino, carboxylic acid, carboxylic acid derivative, C₁₋₁₈alkoxy, C₆₋₁₀aryl or hydroxy groups, a C₆₋₁₀arylC₁₋₆alkyl or C₁₋₆alkylC₆₋₁₀aryl radical, possibly substituted, in particular by one or more amino, carboxylic acid, carboxylic acid derivative, C₁₋₁₈alkoxy, C₆₋₁₀aryl or hydroxy groups; or a nitro function;
or R3 and R4 and/or R4 and R5 form together a ring or a heterocyclic compound, possibly substituted,
X represents a divalent radical, in particular chosen from C₁₋₁₈alkyls, C₂₋₁₈alkenes or C₂₋₁₈alkynes, linear, branched or cyclic, substituted or not, possibly interrupted by a heteroatom,
Y represents a hydrogen atom or a protective group, and
Z represents an amino acid residue, in particular D or L, natural or synthetic, in particular an α, β or γ amino acid residue, the terminal amine function (ie not linked to X) and any lateral chemical functions of Z being protected or not, where Z is linked to the X radical via an amide bond,
in particular, for the compounds described in parts (a), (b) and (c) above, and for the compounds of formula (I) above where A is —O—, —S—, —SO₂—, —C(═S)—, —CO— or a chemical function such that Z′ and X′ are linked by a bioisosteric bond of the amide function, X and X′ are, independently of each other, possibly substituted by one or more chemical functions such as a lateral chain of a natural amino acid; C₁₋₆alkyl; C₂₋₆alkene; C₂₋₆alkyne; C₃₋₈cycloalkyl; C₁₋₆heteroalkyl; C₁₋₆haloalkyl; C₆₋₁₀aryl; C₃₋₁₀heteroaryl; C₅₋₂₀heterocyclic; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, —OC(═NR^{G2})—NR^{G2}C(═NR^{G3})—, —NR^{G2} SO₂—, —NR^{G2}SO₂NR^{G2}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁ heteroalkyl, C₂₋₁₄alkene or C₂₋₁₈alkyne function, linear, branched or cyclic, possibly substituted; or a C₆₋₁₀aryl, C₆₋₁₀heteroaryl, C₅₋₁₀heterocyclic compound, C₁₋₆alkylC₆₋₁₀aryl or C₁₋₆alkylC₆₋₁₀heteroaryl group in which the aryl, heteroaryl or heterocyclic radical is possibly substituted; or, when G represents —NR^{G2}—, R^{G1} and R^{G2} conjointly with the nitrogen atom to which they are bonded form a heterocyclic compound or a heteroaryl, possibly substituted.

2. A compound according to claim 1, having the following formula (II) or one of its pharmaceutically acceptable salts: 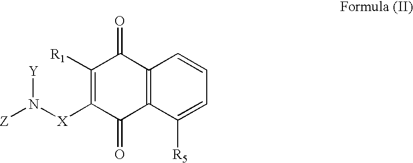
in which
R1 represents a hydrogen atom, an alkyl radical comprising 1 to 6 carbon atoms, or a —(CH₂)ₙ₁—NY′-Z′ group, where
n1 represents an integer number ranging from 1 to 12, in particular from 1 to 6, more especially from 1 to 5, and in particular 1 to 2, and
Y and Y′ represent independently of each other a hydrogen atom or a protective group, and
Z and Z′ represent independently of each other an amino acid residue, in particular D or L, natural or synthetic, in particular an α, β or γ amino acid residue, the terminal amine function (ie not linked to —NY— or —NY′—) and any lateral chemical functions of Z and Z′ being protected or not, where Z and Z′ are linked to the —NY— or —NY′— radical, respectively, via an amide bond;
X represents a —(CH₂)— group, n represents an integer number ranging from 1 to 12, in particular from 1 to 6, more particularly from 1 to 5, and especially from 1 to 2, and
R5 represents a hydrogen atom, a halogen atom or a hydroxyl function, possible substituted.

3. A compound according to claim 2 having formula (II) in which R1 represents an alkyl radical comprising 1 to 6 carbon atoms, especially 1 to 4 carbon atoms, in particular 1 to 2 carbon atoms, or even is the methyl radical.

4. A compound according to claim 3 having formula (II) in which R1 represents a methyl radical and R5 represents a hydrogen atom or a hydroxyl function.

5. A compound according to claim 2 having formula (II) in which R1 represents a hydrogen atom.

6. A compound according to claim 5 having formula (II) in which R1 represents a hydrogen atom and R5 represents a hydroxyl function.

7. A compound according to claim 2 having formula (II) in which R1 represents a —(CH₂)ₙ₁—NY′—COCHRNH₂ group, where
—COCHRNH₂ represents a natural or synthetic amino acid residue, D or L, in which R designates the lateral chain of the said amino acid residue,
n1 represents an integer number ranging from 1 to 5, and in particular from 1 to 2, and
Y′ represents a hydrogen atom or a protective group.

8. A compound according to claim 2 having formula (II) in which R1 represents a —(CH₂)ₙ₁—NY′—COCHRNH₂ group and R5 represents a hydroxyl function.

9. A compound according to claim 2 having one of the following formulae: 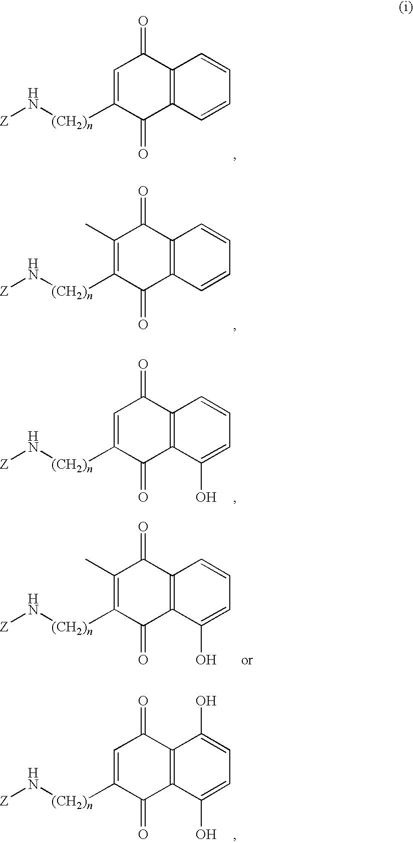
in which n is an integer number ranging from 1 to 12, in particular from 2 to 8, and Z represents an amino acid residue, in particular D or L, natural or synthetic, in particular an α, β or γ amino acid residue, the terminal amine function (ie not linked to —NH—) and any lateral chemical functions of Z being protected or not, where Z is linked to the —NH— radical via an amide bond; 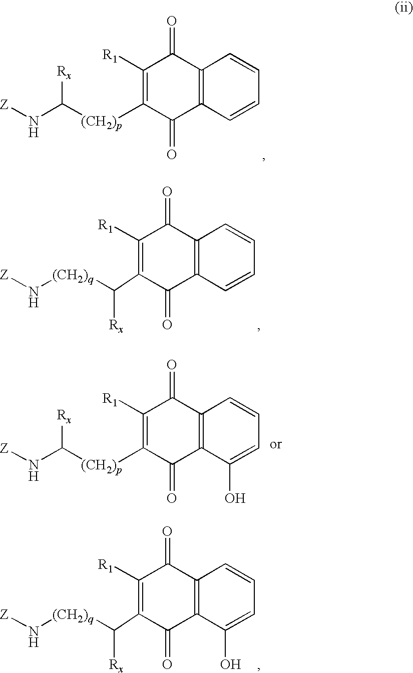
in which Z is as defined above; p and q are independently of each other integers ranging from 0 to 12, in particular from 0 to 7; R1 is as defined in claim 2; and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2} SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)0- or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁₈heteroalkyl, C₂₋₁₈alkene or C₂₋₁₈alkyne function, linear, branched or cyclic, possibly substituted; or a C₆₋₁₀aryl, C₆₋₁₀heteroaryl, C₅₋₁₀heterocyclic compound, C₁₋₆alkylC₆₋₁₀aryl or C₁₋₆alkylC₆₋₁₀heteroaryl group in which the aryl, heteroaryl or heterocyclic radical is possibly substituted, or, when G represents —NR^{G2}—, R^{G1} and R^{G2}, conjointly with the nitrogen atom to which they are linked, form a heterocyclic compound or a heteroaryl, possible substituted; 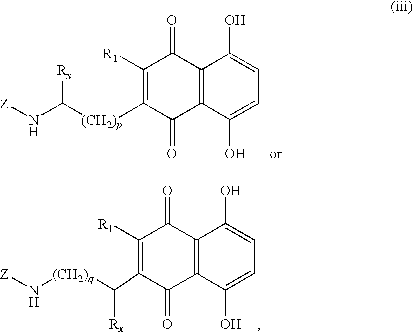
in which Z is as defined above; p and q are independently of each other integers ranging from 0 to 12, in particular from 0 to 7; except when R1 is a hydrogen atom, in which case p and q are independently of each other in integers ranging from 1 to 12, in particular from 1 to 7; R1 is as defined in claim 2; and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; —CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)-—C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —C(═S)NR^{G2}—, NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁₈heteroalkyl, C₂₋₁₈alkene or C₂₋₁₈alkyne function, linear, branched or cyclic, possibly substituted; or a C₆₋₁₀aryl, C₆₋₁₀heteroaryl, C₅₋₁₀heterocyclic compound, C₁₋₆alkylC₆₋₁₀aryl or C₁₋₆alkylC₆₋₁₀heteroaryl group in which the aryl, heteroaryl or heterocyclic radical is possibly substituted, or, when G represents —NR^{G2}—, R^{G1} and R^{G2}, conjointly with the nitrogen atom to which they are linked, form a heterocyclic compound or a heteroaryl, possible substituted; 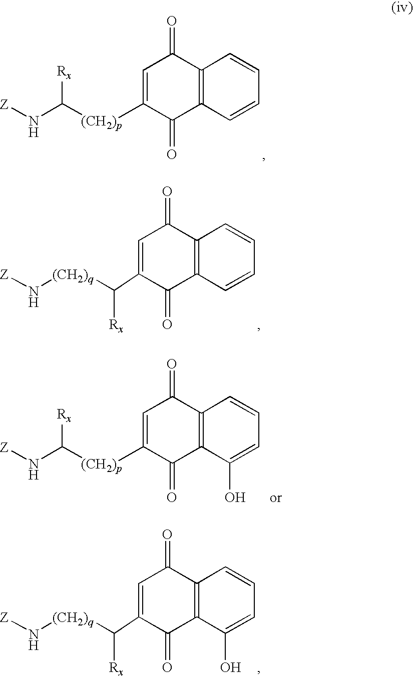
in which Z is as defined above; p and q are independently of each other integers ranging from 0 to 12, in particular from 0 to 7; and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O, —C(═NR^{G2})NR^{G3}—, OC(═NR^{G2})—, —NR^{G2}C(═NR^{G3})—, —NR^{G2} SO₂—, —NR^{G2} SO₂NR^{G3}—, —NR^{G2}C(═S)—, SC(═S)NR^{G2}—NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O, —SC(═O)NR^{G2}—, —NR^{G2}C(═O)S—, —C(═O)S—, —SC(═O)—, —SC(═O)S—, —C(═S)O—, —OC(═S)—, —OC(═S)0- or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁₈heteroalkyl, C₂₋₁₈alkene or C₂₋₁₈alkyne function, linear, branched or cyclic, possibly substituted; or a C₆₋₁₀aryl, C₆₋₁₀heteroaryl, C₅₋₁₀heterocyclic compound, C₁₋₆alkylC₆₋₁₀aryl or C₁₋₆alkylC₆₋₁₀heteroaryl group in which the aryl, heteroaryl or heterocyclic radical is possibly substituted, or, when G represents —NR^{G2}—, R^{G1} and R^{G2}, conjointly with the nitrogen atom to which they are linked, form a heterocyclic compound or a heteroaryl, possible substituted; 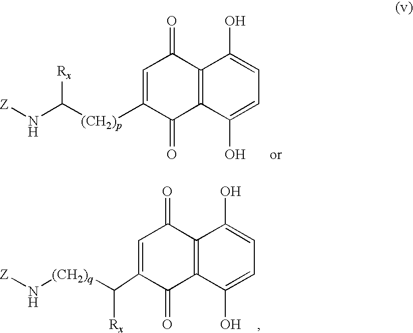
in which Z is as defined above; p and q are independently of each other integers ranging from 1 to 12, in particular from 1 to 7, and Rₓ is a lateral chain of a natural amino acid; is C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}, —OC(═O)—, —NR^{G2}C(═O)—, —OC(═O)0-, —OC(═O)NR^{G2}—, —NR^{G2}C(═O)O—, —NR^{G2}C(═O)NR^{G2}—, —C(═S)-—C(═S)S—, —SC(═S)—, —SC(═S)S—, —C(═NR^{G2})—, —C(═NR^{G2})O, —C(═NR^{G2})NR^{G3}—, OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —C(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁₈heteroalkyl, C₂₋₁₈alkene or C₂₋₁₈alkyne function, linear, branched or cyclic, possibly substituted; or a C₆₋₁₀aryl, C₆₋₁₀heteroaryl, C₅₋₁₀heterocyclic compound, C₁₋₆alkyl C₆₋₁₀aryl or C₁₋₆alkylC₆₋₁₀heteroaryl group in which the aryl, heteroaryl or heterocyclic radical is possibly substituted, or, when G represents —NR^{G2}—, R^{G1} and R^{G2}, conjointly with the nitrogen atom to which they are linked, form a heterocyclic compound or a heteroaryl, possible substituted; 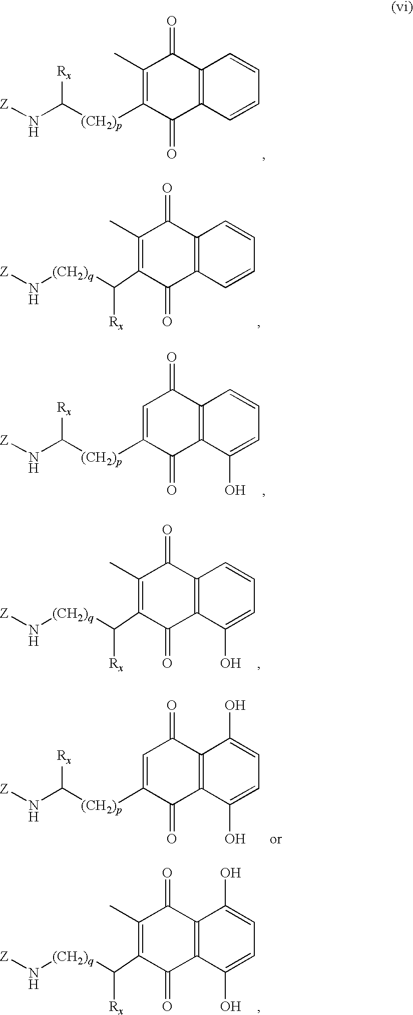
in which Z is defined above; p and q are independently of each other integers ranging from 0 to 12, in particular from 0 to 7, and Rₓ is a lateral chain of a natural amino acid; C₁₋₆alkyl; C₁₋₆heteroalkyl; C₁₋₆haloalkyl; C₆₋₁₀aryl; C₃₋₁₀heteroaryl; C₁₋₆alkylC₆₋₁₀aryl; C₁₋₆alkylC₃₋₁₀heteroaryl; C₁₋₆alkoxy; C₆₋₁₀aryloxy; C₃₋₁₀heteroalkoxy; C₃₋₁₀heteroaryloxy; C₁₋₆heteroalkylthio; C₆₋₁₀arylthio; C₁₋₆heteroalkylthio; C₃₋₁₀heteroarylthio; F; Cl; Br; I; —NO₂; —CN; —CF₃; —CH₂CF₃; —CHCl₂, —CH₂OH; —CH₂CH₂OH; CH₂NH₂; —CH₂SO₂CH₃ or a -GR^{G1} function in which G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)0-, —OC(═O)NR^{G2}, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}S₂NR^{G3}—, —NR^{G2}C(═S)—, —SC(═S)NR^{G2}—, —NR^{G2}C(═S)S—, —NR^{G2}C(═S)NR^{G2}—, —SC(═NR^{G2})—, —C(═S)NR^{G2}—, —OC(═S)NR^{G2}—, NR^{G2}C(═S)O—, —SC(═O)NR^{G2}—, —NR^{G2}C(═O)S—, —C(═O)S—, —SC(═O), —SC(═O)S—, —C(═S)O—, —OC(═S)—, —OC(═S)O— or —SO₂NR^{G2}—, where each occurrence of R^{G1}, R^{G2} and R^{G3} is independently of the other occurrences of R^{G1} a hydrogen atom; a halogen atom; or a C₁₋₁₈alkyl, C₁₋₁₀heteroalkyl, C₂₋₁₈alkene or C₂₋₁₈alkyne function, linear, branched or cyclic, possibly substituted; or a C₆₋₁₀aryl, C₆₋₁₀heteroaryl, C₅₋₁₀-heterocyclic compound, C₁₋₆alkylC₆₋₁₀aryl or C₁₋₆alkylC₆₋₁₀heteroaryl group in which the aryl, heteroaryl or heterocyclic radical is possibly substituted, or, when G represents —NR^{G2}—, R^{G1} and R^{G2}, conjointly with the nitrogen atom to which they are linked, form a heterocyclic compound or a heteroaryl, possible substituted; 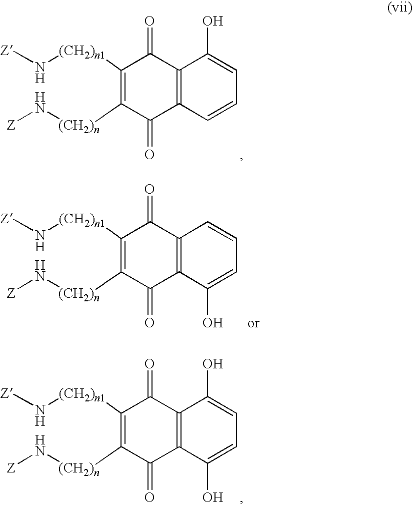
in which n and n1 are independently of each other integers ranging from 1 to 12, in particular from 1 to 5; and Z and Z′ are independently of each other an amino acid residue, in particular D or L, natural or synthetic, in particular an α, β or γ amino acid residue, the terminal amine function (ie not linked to —NH—) and any lateral chemical functions of Z/Z′ being protected or not, where Z and Z′ are linked to the radical —NH— via an amide bond.

10. A pharmaceutical composition comprising, by way of active agent, at least one compound as defined according to claim 1 to 9 in a pharmaceutically acceptable carrier.

11. A method for treating at least one disease involving abnormal cell proliferation, in particular cancer, and in particular a cancer chosen from pancreatic cancer, cancers of the oropharynx, stomach cancer, cancer of the oesophagus, colon and rectal cancer, brain tumours, in particular gliomers, ovarian cancer, liver cancer, kidney cancer, cancer of the larynx, thyroid cancer, lung cancer, bone cancer, multiple myelomas, mesotheliomas and melanomas, skin cancer, breast cancer, prostate cancer, bladder cancer, cancer of the uterus, testicular cancer, non-Hodgkin's lymphoma, leukaemia, Hodgkin's disease and soft-tissue cancers, as well as secondary locations metastatic of the aforementioned cancers, comprising administrating to a subject in need thereof, a pharmaceutically effective amount of a compound of claim 1.

12. Compound of claim 10, wherein the compound is a pro-apoptotic and/or anti-proliferative agent.
